(19) United States
(12) Patent Application Publication
(10) Pub. No.: US 2025/0241957 A1
(43) Pub. Date: Jul. 31, 2025

(54) INTRA-ERYTHROCYTE DEXAMETHASONE FOR TREATMENT OF ATAXIA-TELANGIECTASIA

(71) Applicant: Quince Therapeutics S.p.A., Bresso (IT)

(72) Inventor: Giovanni Mambrini, Bresso (IT)

(73) Assignee: Quince Therapeutics S.p.A., Bresso (IT)

(21) Appl. No.: 19/025,657

(22) Filed: Jan. 16, 2025

Related U.S. Application Data
(60) Provisional application No. 63/625,213, filed on Jan. 25, 2024.
(60) Provisional application No. 63/626,398, filed on Jan. 29, 2024.

(51) Int. Cl. A61K 35/18; A61K 9/00; A61P 25/00

(52) U.S. Cl. CPC A61K 35/18; A61K 9/0019; A61P 25/00

(57) ABSTRACT

The present disclosure is directed to treating patients with Ataxia-Telangiectasia by administering to the subject at least 5 mg of dexamethasone sodium phosphate (DSP) that has been loaded into erythrocytes. Also disclosed herein are methods of treating a pediatric subject with Ataxia-Telangiectasia who is between 9 and 15 kilograms in weight.

### CROSS-REFERENCE TO RELATED PATENT APPLICATIONS

This application claims the benefit under 35 U.S.C. § 119(e) of U.S. Provisional Application No. 63/625,213, filed Jan. 25, 2024, and U.S. Provisional Application No. 63/626,398 filed Jan. 29, 2024, the entire contents of which are incorporated herein by reference.

### BACKGROUND

The present disclosure is directed to intra-erythrocyte loaded dexamethasone for treatment of patients with Ataxia-Telangiectasia.

Ataxia-telangiectasia (A-T) is an autosomal recessive multisystem disorder caused by biallelic pathogenic variants in the Ataxia Telangiectasia Mutated (ATM) gene, located on chromosome 11q22·3-23·1. (Gatti R et al., Nature 1988;336:577-80.) The ATM gene codes for the protein of the same name (ATM) a member of the phosphoinositide 3-kinase-related kinases (PIKKs) which phosphorylates hundreds of proteins important in regulation of DNA repair, RNA-splicing, redox homeostasis, mitochondrial metabolic signaling, oxidative damage, and many other cellular functions. (Lee J-H, Paull T T. Nat Rev Mol Cell Biol. 2021;22:796-814. doi:10.1038/s41580-021-00394-2. Epub 2021 Aug. 24.) ATM gene mutations lead to a complex clinical phenotype with substantial variability in the severity of clinical features. The most common clinical features of A-T include neurological symptoms (ataxia with progressive cerebellar degeneration, involuntary movements, oculomotor apraxia, dysarthria), ocular and cutaneous telangiectasias, repeated infections contributing to chronic lung disease, delayed growth and pubertal development, and predisposition to cancers due to genetic instability and radiation sensitivity. (Rothblum-Oviatt C et al, Orphanet J Rare Dis 2016;11 (1):159. doi:10.1186/s13023-016-0543-7; Petley E et al., PLoS One 2022;17(3):e0264177. doi:10.1371/journal.pone.0264177; and Amirifar P et al., Pediatric Allergy and Immunology 2019;30:277-88.) Laboratory manifestations include abnormal T-cell receptor-ß repertoires, elevated alpha-fetoprotein (AFP) in the majority of affected individuals, lymphopenia in half, and immunoglobulin IgA and IgG2-subclass deficiency in approximately one third. (Zielen S et al., J Clin Immunol. 2021;41:1878-92.) Cancer develops in a quarter of individuals with A-T at a median age of 12.5 years and contributes to their shortened life span. (Suarez F et al., J Clin Oncol. 2014;33:202-8.) Currently, there are no effective therapies to slow disease progression or limit development of systemic disease in individuals with A-T.

In 2006, Italian clinicians observed significant improvement in neurological symptoms of a 3-year-old child with A-T who was given betamethasone for asthma, which prompted a single-participant trial confirming that observation. (Buoni S et al., Arch Neurol. 2006;63:1479-82.) Subsequent studies described a benefit of low-dose oral betamethasone to ameliorate neurological symptoms in children with A-T. However, treatment discontinuation was accompanied by the reappearance of symptoms, suggesting that long-term administration of steroids may be required to preserve the benefit. (Broccoletti T et al., Eur J Neurol. 2008;3:223-8; Broccoletti T et al., Eur J Neurol. 2011;4:564-70; and Zannolli R et al., Mov Disord. 2012;27:1312-6.) In a study of betamethasone used for two years in six participants with A-T, transient neurologic improvement was documented in five out of six participants, but after two years of treatment the improvement persisted in only one of them. Adrenal suppression was documented in all participants. (Hasegawa S et al., Pediatr Neurol. 2019:100:60-66.) These trials indicated that prolonged use of oral steroids was not a viable option for treatment of children with A-T.

### SUMMARY OF THE DISCLOSURE

An aspect of the disclosure is directed to a method for treating Ataxia Telangiectasia in a subject less than 10 years of age, comprising, or alternatively consisting essentially of, or yet further consisting of administering to the subject at least 5 mg of dexamethasone sodium phosphate (DSP) that has been loaded into erythrocytes.

Another aspect of the disclosure is directed to a method for treating Ataxia Telangiectasia in a subject, comprising, or alternatively consisting essentially of, or yet further consisting of administering to the subject at least 5 mg of dexamethasone sodium phosphate (DSP) that has been loaded into erythrocytes.

In some embodiments, the subject is at least 6 years of age. In some embodiments, the subject is less than 10 years of age. In some embodiments, the subject is between 6 and 10 years of age.

In some embodiments, the subject is administered between about 5 mg and about 30 mg DSP. In some embodiments, the subject is administered between about 5 mg and about 22 mg DSP. In some embodiments, the subject is administered about 8 mg or about 22 mg DSP. In some embodiments, the subject is administered about 8 mg or about 17 mg DSP.

In some embodiments, the subject is administered the DSP monthly, every 5 weeks, every 6 weeks, every 7 weeks or every two months.

In some embodiments, the DSP is administered by infusion. In some embodiments, the treatment period is 3 months, 6 months, 9 months, or 12 months.

In some embodiments, the erythrocytes are autologous to the subject.

In some embodiments, the erythrocytes are loaded with DSP by:
(a) removing whole blood from the subject;
(b) using the whole blood, preparing a population of erythrocytes by
   (i) isolating erythrocytes from the whole blood;
   (ii) swelling the erythrocytes using a first hypotonic solution;
   (iii) further swelling the erythrocytes in step (ii) with a second hypotonic solution that is more hypotonic that the first hypotonic solution, wherein the further swelling is done without reaching lysis;
   (iv) concentrating the erythrocytes obtained in step (iii);
   (v) placing the concentrated erythrocytes in contact with a lysing solution comprising, or alternatively consisting essentially of, or yet further consisting of DSP; and
   (vi) adding a sealing solution for the purpose of obtaining a population of erythrocytes loaded with said one or more pharmaceutical products.

In some embodiments, the erythrocytes are loaded with DSP by:
(i) swelling erythrocytes, optionally isolated from whole blood, using a first hypotonic solution;
(ii) further swelling the erythrocytes in step (i) with a second hypotonic solution that is more hypotonic that the first hypotonic solution, wherein the further swelling is done without reaching lysis;
(iii) concentrating the erythrocytes obtained in step (ii);
(iv) placing the concentrated erythrocytes in contact with a lysing solution comprising, or alternatively consisting essentially of, or yet further consisting of DSP; and
(v) adding a sealing solution and thereby obtaining a population of erythrocytes loaded with DSP.

In some embodiments, the erythrocytes are loaded with DSP by: obtaining between 20-100 mL of whole blood of the subject; separating erythrocytes from the whole blood; swelling the erythrocytes using a first hypotonic saline solution having an osmolality between 230 and 150 mOsm/kg; further swelling the erythrocytes using a second hypotonic solution having a lower osmolality than the first hypotonic saline solution, wherein the further swelling is done without reaching lysis, thereby providing swollen erythrocytes; contacting the swollen erythrocytes with a solution comprising, or alternatively consisting essentially of, or yet further consisting of between 2-10 mL of a solution comprising, or alternatively consisting essentially of, or yet further consisting of DSP at a concentration of 25 mg/mL; and incubating the swollen erythrocytes with a hypertonic sealing solution comprising, or alternatively consisting essentially of, or yet further consisting of phosphate-inosine-glucose-pyruvate-adenine (PIGPA).

In some embodiments, the erythrocytes are loaded with DSP by: separating erythrocytes from 20-100 mL of whole blood; swelling the erythrocytes using a first hypotonic saline solution having an osmolality between 230 and 150 mOsm/kg; further swelling the erythrocytes using a second hypotonic solution having a lower osmolality than the first hypotonic saline solution, wherein the further swelling is done without reaching lysis, thereby providing swollen erythrocytes; contacting the swollen erythrocytes with a solution comprising, or alternatively consisting essentially of, or yet further consisting of between 2-10 mL (e.g., 2, 3, 4, 5, 6, 7, 8, 9, 10 mL or any value therebetween) of a solution comprising, or alternatively consisting essentially of, or yet further consisting of DSP at a concentration of 25 mg/mL; and incubating the swollen erythrocytes with a hypertonic sealing solution comprising, or alternatively consisting essentially of, or yet further consisting of phosphate-inosine-glucose-pyruvate-adenine (PIGPA).

In some embodiments, the second hypotonic solution brings the intact erythrocytes to an osmolality between 200 and 170 mOsm/Kg.

In some embodiments, about 50 mL of the whole blood is obtained from the subject.

In some embodiments, about 30 mL of the whole blood is obtained from the subject.

In some embodiments, the subject is between 9 and 15 kilograms in weight.

### BRIEF DESCRIPTION OF THE DRAWINGS

FIG. 1 summarizes disposition of participants including rationale for discontinuations from the study. COVID-19=Coronavirus 2019, ITT=Intention to Treat (all patients who received at least one dose of study medication), mITT=modified Intention to Treat (all patients who received study drug and had at least one efficacy assessment). Per protocol population=patients who missed<1 study treatment and completed treatment within 228 days.

FIGS. 2A-2D provide a summary of the study's main efficacy results. Primary endpoint (A-B) mICARS changes from Baseline up to month 6; Key secondary endpoint (C-D) Percent of patients showing an improvement in CGI-C score at month 6. (A-B) The delta (Δ) represents the change in mICARS score, from baseline to month 6, for the entire mITT population (A) and for the 6 to 9year-olds (B). The p value refers to the comparison between the high dose EryDex and placebo groups. (C-D) The CGI-C outcomes: histograms represent the percentage of patients with improved score in clinical global impression of disease at 6 months.

FIGS. 3A-3B show disease progressing in A-T patients. (A) Graph of disease progression versus age in A-T patients. (B) Natural History Progression of the RmICARS Based on Baseline Data from Ataxia Telangiectasia Subjects.

FIG. 4 shows a non-limiting design for the red cell loader (RCL).

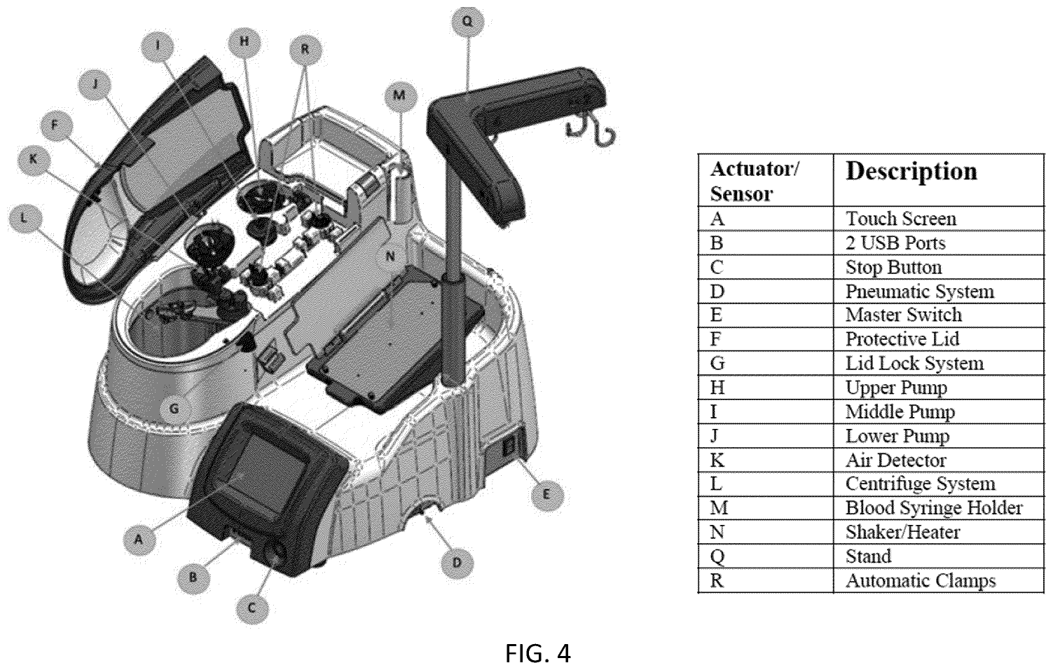

FIG. 5 shows a non-limiting design for the EryKit_01. (1) Cassette (viewed from bottom), (2) Spike-on connector for Hypotonic Solution 1, (3) Spike-on connector for Hypotonic Solution 2, (4) Spike-on connector for the 2 1-litre bags of injectable saline solution (washing), (5) Waste bag line, (6) Blood port line for intake of 30-50 mL of subject's blood (welding with the Syringe Kit), (7) Final bag (to collect EryDex) with satellite bag for EryDex sampling, (8) Waste bag (to collect waste fluid), (9) Transfer bag (attaches to the RCL shaker/heater plate), (10) Vacuum pump RCL connector tube (right side of device, stand side), (11) Reservoir with dedicated Hang Label (for ultrafiltrate), (12) Hemofilter with dedicated Hang Label (for red blood cell concentration), (13) Bowl (for blood separation and washing), (14) Injection point (for injecting the drug and PIGPA Hypertonic Solution during the EryDex process).

### DETAILED DESCRIPTION

Ataxia telangiectasia is a multi-system disorder with progressive neurodegeneration being the primary symptom. In patients with classical A-T, comprising 82-97.5% of cases, wheelchair dependence occurs at a median of 10 years of age, while in patients with variant disease, wheelchair dependence occurs at a median of 26-27 years. (Petley E et al., PLoS One 2022;17(3):e0264177. doi:10.1371/journal.pone. 0264177.) Delaying neurologic progression is paramount to improve A-T patients' quality of life. Studies performed in mice or in cell lines suggested several pathways leading to neuronal injury in A-T including increased oxidative stress from sustained microglial activation, increased inflammation, mitochondrial exhaustion, and altered neuronal membrane polarization and neurotransmitter levels. (Bourseguin J et al., Nucleic Acid Res. 2022;50:2700-18; Aguado J et al., Aging Cell. 2021 September;20(9):e13468. doi:10.1111/acel.13468; Stern N et al., J Biol Chem. 2002;277:602-8; and Kirshner M et al., J Mol Neurosci. 2012;46:554-68.)

There are no curative or approved therapies for A T; however, many agents including nicotinamide riboside, alpha-lipoic acid, triheptanoin, acetyl-DL-leucine, amantadine sulfate, and myo-inositol have been studied in hope of ameliorating oxidative damage, inflammation, mitochondrial dysfunction, and neurological symptoms. Promising initial results with some of these therapies require confirmation in randomized trials. (Kuhn K et al., Expert Opin Investig Drugs 2023;32:693-704.) The investigational use of EryDex for A-T started in 2010, and the favorable results of the phase 2 study prompted this study. The primary analysis from the current study indicated that children 6-9 years of age benefit from this treatment as they showed statistically significant improvement in neurologic exam and change in global impression of disease severity.

### Definitions

As it would be understood, the section or subsection headings as used herein is for organizational purposes only and are not to be construed as limiting and/or separating the subject matter described.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods, devices, and materials are now described. All technical and patent publications cited herein are incorporated herein by reference in their entirety. Nothing herein is to be construed as an admission that the disclosure is not entitled to antedate such disclosure by virtue of prior disclosure.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3rd edition (Cold Spring Harbor Laboratory Press (2002)); Sohail (ed.) (2004) Gene Silencing by RNA Interference: Technology and Application (CRC Press).

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compounds, agents, compositions and methods include the recited elements, but not exclude others. “Consisting essentially of” when used to define compounds, agents, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” In some embodiments, there is a proviso that the terms are not preceded by the term “about.” It also is to be understood, although not always explicitly stated, that the reagents described herein are merely exemplary and that equivalents of such are known in the art.

The term “about,” as used herein when referring to a measurable value such as an amount or concentration and the like, is meant to encompass variations of 20%, 10%, 5%, 1%, 0.5%, or even 0.1% of the specified amount. As it relates to age, the term “about” encompasses within 1-3 months of the stated age (age±1, ±2, ±3 months, or any value therebetween). As it relates to weight, the term “about” encompasses within 1-5 kilograms of the stated weight (weight in kilograms±1, ±2, ±3, ±4, ±5 kilograms, or any value therebetween). As it relates to time, the term “about” encompasses within 1-5 months of the stated time (time in months±1, ±2, ±3, ±4, ±5 kilograms, or any value therebetween).

As used herein, comparative terms as used herein, such as high, low, increase, decrease, reduce, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference. In some embodiments, such variation can refer to about 1%, or about 2%, or about 3%, or about 4%, or about 5%, or about 6%, or about 7%, or about 8%, or about 0%, or about 10%, or about 20%, or about 30%, or about 40%, or about 50%, or about 60%, or about 70%, or about 75%, or about 80%, or about 85%, or about 90%, or about 95%, or about 96%, or about 97%, or about 98%, or about 99% of the reference.

As will be understood by one skilled in the art, for any and all purposes, all ranges disclosed herein also encompass any and all possible subranges and combinations of subranges thereof. Furthermore, as will be understood by one skilled in the art, a range includes each individual member.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

The terms or “acceptable,” “effective,” or “sufficient” when used to describe the selection of any components, ranges, dose forms, etc. disclosed herein intend that said component, range, dose form, etc. is suitable for the disclosed purpose.

A “composition” is intended to mean a combination of active agent and another compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A composition as disclosed herein can be a pharmaceutical composition. A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

A combination as used herein intends that the individual active ingredients of the compositions are separately formulated for use in combination and can be separately packaged with or without specific dosages. The active ingredients of the combination can be administered concurrently or sequentially.

An “effective amount” is an amount sufficient to effect beneficial or desired results. An effective amount can be administered in one or more administrations, applications, or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific agent employed, bioavailability of the agent, the route of administration, the age of the animal and its body weight, general health, sex, the diet of the animal, the time of administration, the rate of excretion, the drug combination, and the severity of the particular disorder being treated and form of administration. In general, one will desire to administer an amount of the agent that is effective to achieve a serum level commensurate with the concentrations found to be effective in vivo. These considerations, as well as effective formulations and administration procedures are well known in the art and are described in standard textbooks.

“Therapeutically effective amount” of an agent refers to an amount of the agent that is an amount sufficient to obtain a pharmacological response; or alternatively, is an amount of the agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses. Thus, a therapeutically effective amount may be administered in one or more administrations.

As used herein, the phrase “derived from” means isolated from, purified from, or engineered from, or any combination thereof.

The term “subject,” “host,” “individual,” and “patient” are as used interchangeably herein to refer to animals, typically mammalian animals. Any suitable mammal can be treated by a method described herein. In some embodiment, the subject is a human pediatric subject. In some embodiments, the subject is less than 10 years old. In some embodiments, the subject is 6 years old or older and less than 10 years old.

As used herein, the term “autologous,” in reference to cells refers to cells that are isolated and infused back into the same subject (recipient or host). “Allogeneic” refers to non-autologous cells.

As used herein, the term “encapsulate” means enclose a substance (e.g., a drug) within a membrane structure (e.g., a cell, an erythrocyte).

### EryDex System (EDS) Process (or Procedure)

In some embodiments, the EDS Process (or Procedure) is used to encapsulate DSP in/load DSP into autologous erythrocytes, by using the EDS and its components.

Briefly, about 20-60 mL (e.g., about 20, 25, 30, 35, 40, 45, 50, 55, 60 mL or any value therebetween) of blood is processed with hypotonic saline solutions to permit temporary reduction of osmolarity that allows the diffusion of the DSP into the cells. Subsequent addition of a Hypertonic Solution (PIGPA) allows for restoration of physiological osmolarity levels and entrapment of DSP within the RBC. The drug-loaded RBCs are extensively washed to remove extracellular content (products of cell lysis, process solutions and non-encapsulated DSP) automatically by the red cell loader (RCL) and then prepared in the final bag (dexamethasone sodium phosphate (DSP) into erythrocytes—EryDex) ready for infusion into the same subject/patient. The bag containing erythrocytes is removed from the apparatus, as the infusion process does not require contact with the EryKit_01. The reinfusion requires a suitable transfusion set with an in-line micro-aggregate filter.

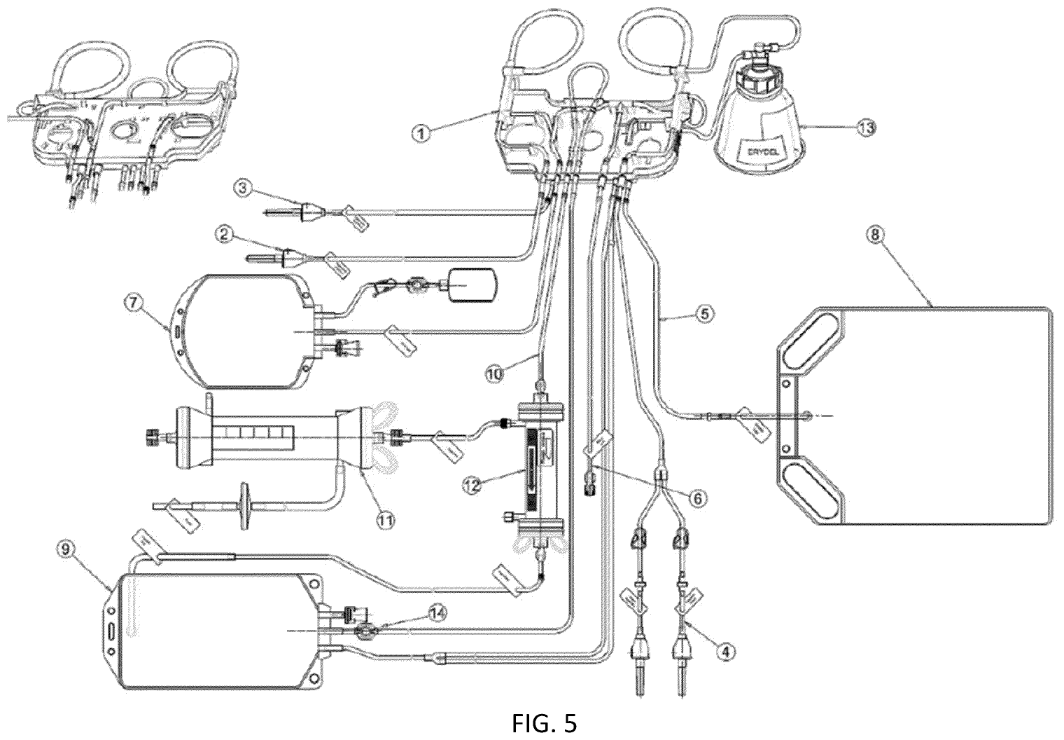

As used herein, the term “EryKit_01” refers to a sterile and non-pyrogenic, single-use, medical device consisting of a closed system made of tubes (or lines), bags, filters (including an hemofilter), ports, centrifuge bowl, and other connecting elements. EryKit_01, when assembled on the EDS, provides a fully contained system that enables the required blood, drug, and processing solutions to be processed and treated in a controlled and stepwise manner. The tubing and components of the EryKit_01 are arranged together in a way that enables the phases of the EDS process to be automated by the RCL. For ease of use, the EryKit_01 is provided to the user with the tubing and components completely assembled. A non-limiting example EryKit_01 is shown in FIG. 5. EryKit_01 is manufactured by Quince Therapeutics.

The RCL controls all phases of the procedure by monitoring all parameters and by automatically checking the operation of the whole system. The automatic sensors inform the user of any abnormal system conditions. The equipment includes a user interface that allows operator interaction through a touch screen. As the operator is not allowed to change any procedure parameter, the touch screen is mainly informative. The RCL is equipped with internal control electronics distributed over 3 microprocessor cards. The main actuator sensors on the machine are: centrifuge unit that can reach a rotation speed of 5600 rpm to allow the separation of RBCs from blood components and to perform washing using saline; 3 peristaltic pumps used by the machine for moving liquid during the process; 2 automatic clamps units, each of which allow groups to inject the liquid flow in 3 different directions; 2 load cells on the stand to permit weighing of liquids and in general to handle the mass balance of fluids; shaker unit for mixing liquids. This unit is equipped with a hot plate able to control the agitation at temperatures different from ambient hydraulic staff that allows control of negative and positive air pressure through a pump and a series of valves.

The EryDex procedure ensures a rapid, short-lived peak, followed by low, slowly declining serum drug concentrations for approximately 4 weeks, avoiding the need for daily administration of higher oral doses of systemic steroids. The enhanced penetration of the central nervous system (CNS) by dexamethasone compared with prednisolone, together with the low plasma exposure associated with the EryDex suggests that this method of administration may be associated with beneficial effects without the risk of steroid side effects.

Pharmacokinetics (PK) is the only characteristic of the drug product (DSP) pharmacology that differs from that obtained with the ordinary routes of administration (oral, IV, intramuscular, etc.) due to the novel method of administration by ex vivo encapsulation of the drug into human autologous RBCs which are then infused. The PK of the drug product in humans was evaluated in clinical trials. It is not possible to study the PK in animal models because the dephosphorylation rate of the pro-drug, DSP, to the diffusible active drug, dexamethasone, in human RBCs is very different from RBC dephosphorylation rates in other species (Zocchi E. et al., Advances in the Biosciences (Volume 81), Pergamon Press, R. Green, J. R. DeLoach (1991)).

### Therapy Outcomes

As used herein, the therapy outcome (aka. “the primary efficacy endpoint”) for the disclosed therapy is a change in neurological symptoms as compared to the baseline (before the treatment). In some embodiments, the therapy outcome is measured by comparing the DSP treatment to the placebo control. In some embodiments, neurological symptoms of the subjects are assessed based on videotaped exams. In some embodiments, neurological symptoms are scored by the International Cooperative Ataxia Rating Scale (ICARS) which includes assessment of posture and gait, kinetic functions, speech, and oculomotor function. In some embodiments, ICARS is as defined in Trouillas P et al. (J Neurol Sci 1997;145:205-11). In some embodiments, neurological symptoms are scored by the modified ICARS, which consists of 11 items across three domains (posture and gait, kinetic function, and speech) while eliminating oculomotor domain and some items from the kinetic domain. The mICARS score is rated between 0-54 (higher scores indicate worse neurologic function), as compared to 0-100 in ICARS, and the modification was done to focus on domains that better predict motor functioning and are more relevant when evaluating disease progression in A-T patients. Individual assessment points for ICARS and mICARS are listed in Table 4.

### Modes for Carrying out the Disclosure

### Methods for Treating A-T

Another aspect of the disclosure is directed to a method for treating Ataxia Telangiectasia in a subject, comprising, or alternatively consisting essentially of, or yet further consisting of administering to the subject at least 5 mg of dexamethasone sodium phosphate (DSP) that has been loaded into erythrocytes.

An aspect of the disclosure is directed to a method for treating Ataxia Telangiectasia in a subject less than 10 years of age, comprising, or alternatively consisting essentially of, or yet further consisting of administering to the subject at least 5 mg of dexamethasone sodium phosphate (DSP) that has been loaded into erythrocytes. In some embodiments, the at least 5 mg of DSP is loaded into a single batch of erythrocytes, which are administered into the subject. Over time DSP is dephosporylated by the erythrocytes and become dexamethasone (the active drug), which diffuses out of the erythrocytes.

In some embodiments, the subject is at least 6 years of age and less than 10 years of age (e.g., about 6, 7, 8, 9, or 10 years old).

In some embodiments, the subject is administered between about 5 mg and about 30 mg DSP (e.g., 4, 5, 6, 7, 8, 9, 10, 11, 12, 13, 14, 15, 16, 17, 18, 19, 20, 21, 22, 23, 24, 25, 26, 27, 28, 29, 30 mg, or any value therebetween) that has been loaded into erythrocytes.

In some embodiments, the subject is administered between about 5 mg and about 22 mg DSP (e.g., 4, 5, 6, 7, 8, 9, 10, 11, 12, 13, 14, 15, 16, 17, 18, 19, 20, 21, 22, 23, 24 mg, or any value therebetween) that has been loaded into erythrocytes.

In some embodiments, the subject is administered about 8 mg or about 22 mg DSP (e.g., 7, 8, 9, 10, 11, 12, 13, 14, 15, 16, 17, 18, 19, 20, 21, 22, 23, 24 mg, or any value therebetween) that has been loaded into erythrocytes.

In some embodiments, the subject is administered about 8 mg or about 17 mg DSP (e.g., 7, 8, 9, 10, 11, 12, 13, 14, 15, 16, 17, 18, 19 mg, or any value therebetween) that has been loaded into erythrocytes.

In some embodiments, the subject is administered the erythrocyte-loaded DSP monthly, every three weeks or every other week. In a specific embodiment, the subject is administered the DSP that has been loaded into erythrocytes monthly, every 5 weeks, every 6 weeks, every 7 weeks or every two months.

In some embodiments, the DSP that has been loaded into erythrocytes is administered by infusion. As used herein, “infusion” refers to the process of transferring blood products (e.g., erythrocytes loaded with DSP) into a person's circulation intravenously.

As used herein, the phrase “treatment period” refers to the time period during which the subject receives the disclosed treatment. In some embodiments, the treatment period begins with the administration of the first treatment and ends with the administration of the last treatment.

In some embodiments, the treatment period is between about 2 and about 15 months (e.g., about 2, 3, 4, 5, 6, 7, 8, 9, 10, 11, 12, 13, 14, 15 months, or any value therebetween). In some embodiments, the treatment period is about 3 months, about 6 months, about 9 months, or about 12 months.

In some embodiments, the subject is treated for between about 2 and about 15 months (e.g., about 2, 3, 4, 5, 6, 7, 8, 9, 10, 11, 12, 13, 14, 15 months, or any value therebetween). In some embodiments, the subject is treated for about 3 months, about 6 months, about 9 months, or about 12 months.

In some embodiments, the time period between the first treatment and the last treatment is between about 2 and about 15 months (e.g., about 2, 3, 4, 5, 6, 7, 8, 9, 10, 11, 12, 13, 14, 15 months, or any value therebetween). In some embodiments, the time period between the first treatment and the last treatment is about 3 months, about 6 months, about 9 months, or about 12 months.

In some embodiments, the erythrocytes are autologous to the subject.

In some embodiments, the erythrocytes are loaded with DSP by:
(i) swelling erythrocytes, optionally isolated from whole blood, using a first hypotonic solution;
(ii) further swelling the erythrocytes in step (i) with a second hypotonic solution that is more hypotonic that the first hypotonic solution, wherein the further swelling is done without reaching lysis;
(iii) concentrating the erythrocytes obtained in step (ii);
(iv) placing the concentrated erythrocytes in contact with a lysing solution comprising, or alternatively consisting essentially of, or yet further consisting of DSP; and
(v) adding a sealing solution and thereby obtaining a population of erythrocytes loaded with DSP.

In some embodiments, the erythrocytes are loaded with DSP by:
(a) removing whole blood from the subject;
(b) using the whole blood, preparing a population of erythrocytes by
   (i) isolating erythrocytes from the whole blood;
   (ii) swelling the erythrocytes using a first hypotonic solution;
   (iii) further swelling the erythrocytes in step (ii) with a second hypotonic solution that is more hypotonic that the first hypotonic solution, wherein the further swelling is done without reaching lysis;
   (iv) concentrating the erythrocytes obtained in step (iii);
   (v) placing the concentrated erythrocytes in contact with a lysing solution comprising, or alternatively consisting essentially of, or yet further consisting of DSP; and
   (vi) adding a sealing solution and thereby obtaining a population of erythrocytes loaded with DSP.

In some embodiments, the erythrocytes are loaded with DSP by: separating erythrocytes from 20-100 mL of whole blood; swelling the erythrocytes using a first hypotonic saline solution having an osmolality between 230 and 150 mOsm/kg; further swelling the erythrocytes using a second hypotonic solution having a lower osmolality than the first hypotonic saline solution, wherein the further swelling is done without reaching lysis, thereby providing swollen erythrocytes; contacting the swollen erythrocytes with a solution comprising, or alternatively consisting essentially of, or yet further consisting of between 2-10 mL (e.g., 2, 3, 4, 5, 6, 7, 8, 9, 10 mL or any value therebetween) of a solution comprising, or alternatively consisting essentially of, or yet further consisting of DSP at a concentration of 25 mg/mL; and incubating the swollen erythrocytes with a hypertonic sealing solution comprising, or alternatively consisting essentially of, or yet further consisting of phosphate-inosine-glucose-pyruvate-adenine (PIGPA).

In some embodiments, the erythrocytes are loaded with DSP by: obtaining between 20-100 mL of whole blood of the subject; separating erythrocytes from the whole blood; swelling the erythrocytes using a first hypotonic saline solution having an osmolality between 230 and 150 mOsm/kg; further swelling the erythrocytes using a second hypotonic solution having a lower osmolality than the first hypotonic saline solution, wherein the further swelling is done without reaching lysis, thereby providing swollen erythrocytes; contacting the swollen erythrocytes with a solution comprising, or alternatively consisting essentially of, or yet further consisting of between 2-10 mL (e.g., 2, 3, 4, 5, 6, 7, 8, 9, 10 mL or any value therebetween) of a solution comprising, or alternatively consisting essentially of, or yet further consisting of DSP at a concentration of 25 mg/mL; and incubating the swollen erythrocytes with a hypertonic sealing solution comprising, or alternatively consisting essentially of, or yet further consisting of phosphate-inosine-glucose-pyruvate-adenine (PIGPA).

In some embodiments, the second hypotonic solution brings the intact erythrocytes to an osmolality between 200 and 170 mOsm/Kg (e.g., 200, 195, 190, 185, 180, 175, or 170 mOsm/Kg, or any value therebetween).

In some embodiments, about 50 mL (e.g., 45, 46, 47, 48, 49, 50, 51, 52, 53, 54, 55 mL, or any value therebetween) of the whole blood is obtained from the subject.

In some embodiments, about 30 mL (e.g., 25, 26, 27, 28, 29, 30, 31, 32, 33, 34, 35 mL, or any value therebetween) of the whole blood is obtained from the subject.

In some embodiments, the subject is between about 9 and about 15 kilograms (e.g., 8, 9, 10, 11, 12, 13, 14, 15, 16, or any value therebetween) in weight.

In some embodiments, the erythrocytes of the subject are loaded using an erythrocyte-drug loading system process (e.g., the EryDex system process).

In some embodiments, upon treatment, the subject shows improvement in at least one of posture and gait, kinetic function, speech, and oculomotor function. In some embodiments, the subject's improvement upon treatment is measured by International Cooperative Ataxia Rating Scale (ICARS). In some embodiments, upon treatment, the subject shows improvement in at least one of posture and gait, kinetic function, and speech. In some embodiments, the subject's improvement upon treatment is measured by a modified International Cooperative Ataxia Rating Scale (mICARS).

### EXAMPLES

### Example 1: The ATTeST trial

The encapsulation of dexamethasone sodium phosphate (DSP) into autologous erythrocytes (EryDex) was developed as a novel option for continuous delivery of steroids to patients who require prolonged use. After an initial short-lived peak, dexamethasone is released slowly from erythrocytes over a four-week period, avoiding side effects seen with daily steroid use. (Mambrini G et al., Int J Pharm 2017;517:175-84.) EryDex was studied in a phase 2 open label-trial in 22 children with A-T, and they showed statistically significant improvement in ICARS compared to baseline, with a mean reduction of four points, after 6 months of treatment. (Chessa L et al., Orphanet J Rare Dis. 2014;9:5. doi:10.1186/1750-1172-9-5.) Treatment was continued in a subgroup of four participants up to 24 months, confirming continuous neurologic response and a favorable safety profile. (Leuzzi V et al., Neurol Neuroimmunol Neuroinflamm. 2015;2:e98. doi:10.1212/NXI.0000000000000098.) Previous EryDex studies provided preliminary dosing data and encouraging safety and efficacy results which prompted this phase 3 study (ATTeST) of EryDex in A-T patients.

The primary objective of the ATTeST trial was to evaluate efficacy of EryDex at two dose levels (LD and HD infusion), compared to placebo, on neurological symptoms of people with A-T. Efficacy was measured by assessing the change in mICARS from baseline to month 6. The key secondary efficacy endpoint was the change in participant's global clinical status from baseline until month 6. This was measured by using the Clinical Global Impression-Change scale (CGI-C). The safety objective was to evaluate the safety and tolerability of two dose levels of EryDex compared to placebo in participants with A-T, based on the occurrence of adverse events. The results of primary efficacy analyses done at month 6 and describe adverse events reported during 12 months of treatment are described herein.

Study design. This was an international, multi-center, randomized, double-blind, placebo-controlled, phase 3 study. The goal of the study was to assess the effect of two non-overlapping doses of EryDex, administered by monthly intravenous (IV) infusion, on the neurological symptoms of people with A-T. The initial treatment period was 6 months. All participants who completed efficacy assessments over the initial 6 months were eligible to continue in an additional 6-month, double-blind, placebo-controlled treatment, designed to collect longer-term safety and efficacy data.

The trial (NCT02770807) was conducted in accordance with the Good Clinical Practice guidelines of the International Conference on Harmonization (ICH) and the ethical principles of the Declaration of Helsinki. Written informed consent/parental permission was obtained from all participants and/or parents or legal guardians, before screening. An independent data and safety monitoring board reviewed the safety data throughout the trial. The protocol, approved by regulatory authorities and ethics committees of all participating study sites is available on clinicaltrials.gov/study/NCT02770807.

Participants. Patients≥6 years of age and≥15 kg, followed at participating study centers, were recruited by site investigators. Eligibility was based on clinical criteria for diagnosis of A-T by neurologic signs including incoordination of hand and eyes in lateral gaze deflection, and gait ataxia, but autonomous gait (ICARS walking score<4) had to be preserved. Genetic testing for A-T was required; however, the diagnosis established based on clinical criteria was sufficient for enrollment and treatment. Exclusion criteria included lymphopenia CD4<400/mm³ for participants 6 years of age, or<150/mm³ for>6-year-olds; history of severe immunodeficiency; severe or unstable pulmonary, hepatic, or renal disease; uncontrolled diabetes; hemoglobinopathies; or current neoplastic disease. Protocol v 10·0 (16 Apr. 2019) modified eligibility criteria related to CD4+ count. Prior to this modification exclusion was CD4<200/mm³ in>6-years-olds. This was done to facilitate patients' enrollment, after observing that CD4+ counts often varied between 150-200/mm³ without an associated problem of infections. A complete list of eligibility criteria are provided in the study protocol clinicaltrials.gov/study/NCT02770807.

Randomization and masking. Participants were randomly assigned to one of the three treatment groups (1:1:1) using an independent Interactive Web Response System (IWRS). A minimization procedure ensured that the proportions of male and female, and younger (6-9 years) and older (≥10 years) participants were comparable across treatment groups. Participants, sponsor, clinical staff, investigators, and central reviewers were masked to treatment assignment and dose allocations. After the 6-month initial treatment period, participants were eligible to continue in an additional 6-month, double-blind, placebo-controlled treatment, designed to collect longer term safety and efficacy data. After the initial 6 months of treatment, participants randomized to one of the two dose levels of EryDex remained in the same treatment arm. Those originally randomized to the placebo group were re-allocated to receive one of the dose levels or remain in the placebo group. All participants who completed treatment on ATTeST study were subsequently eligible to enroll in an open-label extension study (IEDAT-03-2018;OLE-IEDAT) and receive high dose EryDex.

EryDex treatment and dexamethasone sodium phosphate (DSP) dose determination. The EryDex System (EDS), manufactured by EryDel, S.p.A., Medolla, Italy, is classified by the Food and Drug Administration (FDA) as a drug/device combination product and consists of multi-use Red Cell Loader (RCL); a single-use EryKit_01 and a Syringe Kit; three processing solutions (Hypotonic solutions 1 and 2, and a hypertonic solution; and a drug [DSP]). The RCL system automates the 18-step process. Briefly, 50 mL of whole blood obtained from the participant was loaded to RCL and hypotonic saline solutions were added to permit temporary reduction of osmolarity that allowed the diffusion of DSP into the cell. Subsequent addition of a hypertonic solution restored physiological osmolarity levels and entrapped DSP within the red blood cells (RBC). The drug-loaded RBCs were washed automatically by the RCL to remove products of cell lysis, processing solutions, and non-encapsulated DSP. Sterility cultures were obtained and the product was infused to the same participant over 30-50 minutes. In vivo, DSP slowly dephosphorylates (via enzymatic action within the RBC) to dexamethasone, which diffuses through the RBC membrane into the plasma. This delivers dexamethasone at stable, low levels over an extended period of time.

The EDS process was performed at each center by specifically trained personnel. Prior to each EryDex infusion, a sample was collected from the final bag and the concentration of DSP measured (Syneos Health Clinique, 2500 Einstein Street Québec, QC GIP 0A2, Canada). Participants in the LD group received EryDex prepared using 2.0 mL of the 25 mg/mL DSP solution, plus 11 mL sterile water for injection in the same syringe, for a total of 13·0 mL, which delivered 8·23 mg±3·30 mg (mean±standard deviation) of DSP. Participants in the HD group received EryDex which was prepared using 5·0 mL of the 25 mg/mL DSP solution, plus 11 mL sterile water for injection in the same syringe, for a total of 16 mL, which delivered 17·4±5·38 mg (mean±standard deviation) of DSP. The placebo group received autologous erythrocytes treated with a placebo solution (5 mL of 0·372% NaCl solution), plus 11 mL sterile water for injection in the same syringe, for a total of 16 mL. Processing and infusion took 2-3 hours to complete, and were repeated monthly.

Study procedures. Prior to the first EryDex dose, baseline ICARS and CGI evaluations were videotaped. These assessments were repeated at 3, 6, 9, and 12 months. EryDex was administered monthly for 12 doses, with the allowed window between the infusions being 21-31 days. All endpoint efficacy assessments, including participants who discontinued treatment prematurely, were scheduled at the end of the initial treatment period (month 6 visit). Biological materials were also collected according to the protocol for exploratory analyses, which are ongoing.

The COVID-19 pandemic altered the conduct of the study. The enrollment was closed early with 175 randomized participants (176 enrolled), as opposed to the 180 planned, and unanticipated delays and omissions of treatments occurred.

Outcome measures. The primary efficacy endpoint was a change in neurological symptoms from baseline to month 6, measured by the mICARS, and compared EryDex to the placebo control. This was assessed by central raters based on videotaped exams. The International Cooperative Ataxia Rating Scale (ICARS) includes assessment of posture and gait, kinetic functions, speech, and oculomotor function. (Trouillas P et al., J Neurol Sci 1997;145:205-11.) ICARS scale has been validated in children with A-T, 7-14 years of age. (Nissenkorn A et al., Eur J Paediatr Neurol. 2016;20:140-6.) The modified ICARS consists of 11 items across three domains (posture and gait, kinetic function, and speech) while eliminating oculomotor domain and some items from the kinetic domain. The mICARS score is 0-54 (higher scores indicate worse neurologic function), as compared to 0-100 in ICARS, and the modification was done to focus on domains that better predict motor functioning and are more relevant when evaluating disease progression in A-T patients. The use of mICARS as the primary efficacy endpoint was agreed upon in discussion with the FDA and key opinion leaders. The mICARS was derived during analysis from the full ICARS score documented by investigators. The two scales are presented in Table 4.

All ICARS raters were neurologists or neurophysiotherapists who underwent training and met qualification criteria prior to and during the study. Assessments performed by the site rater were videotaped and reviewed remotely by a central rater to minimize inter-rater variability. The central rater's scores were used in the primary analysis, except for speech, and in instances where two different central raters could not score a certain item from the videotaped exam, in which case site rater's score for that item was used.

The key secondary efficacy outcome was the Clinical Global Impression of Change (CGI-C), an assessment tool built for children with A-T as another means of evaluating change in disease severity. (Nissenkorn A et al., Eur J Paediatr Neurol. 2016;20:140-6.) To determine severity of illness by CGI, clinicians conducted an interview and examination assessing the participant's appearance, neurological functioning, activities of daily living, and mood. CGI of disease was described on a scale 0-4 (0=no signs of disease, 1=mild, 2=moderate, 3=severe, 4=very severe disease). Once the severity was determined, the GCI-C scale assesses change in the CGI of severity from baseline, using a 7-point scale, ranging from 1 (very much improved) to 7 (very much worse), with a score of 4 indicating no change. The assessment of CGI-C and mICARS was done by separate raters. All investigators evaluating efficacy endpoints were masked for treatment allocation and did not have access to other raters' efficacy assessments. Safety of treatment was assessed by obtaining a history, physical exam, laboratory tests, and by documenting Treatment-Emergent Adverse Events (TEAEs), serious AEs, and potentially steroid-related AEs.

A list of all other outcome measures, all evaluations done during the trial, and primary and secondary endpoint assessment scales are described in the study protocol. clinicaltrials.gov/study/NCT02770807.

Statistical analysis. Sample size calculations were based on the analysis of the primary efficacy endpoint from the phase 2 study and showed that 54 participants per treatment arm would provide sufficient power for this study. (Chessa L et al., Orphanet J Rare Dis. 2014;9:5. doi:10.1186/1750-1172-9-5.) The estimate was based on a “two repeated measure study design” to assess the change in mICARS from baseline between the LD or HD group and placebo, with a two-sided 0·05 significance level, when the treatment effect ranged between 3·0 and 3·7, and the standard deviation ranged between 5·0 and 7·0 (PASS®, Module “Tests for Two Means in a Repeated Measures Design”).

The populations analyzed were the following: (1) ITT population/Safety Population: all participants who received any amount of randomized treatment; (2) modified ITT (mITT): all randomized participants who received at least one dose of study medication and had at least one post-baseline efficacy assessment of the primary outcome; and (3) Per-Protocol (PP) population: all participants who did not have any major protocol violations and completed the treatment period of the study within 228 days, allowing≤1 missed dose. Participants who prematurely discontinued the investigational product due to AEs, without protocol violations, and returned for their final evaluation, were still part of the PP population. All determinations regarding major protocol violations and exclusions from the PP population were discussed and agreed upon during a blinded data review meeting prior to breaking the treatment blind and commencing the final analysis on the locked database.

The primary efficacy variable, mICARS total score change from baseline to efficacy assessment visits (month 3 and 6), was analyzed using a mixed model repeated measures (MMRM) with the baseline mICARS value as covariate and five fixed effects (treatment, age [6 to 9 years, ≥10 years], sex, region, visit) and one interaction term (treatment-by-visit). The treatment effect, reported as least squares mean (LSM), together with the associated two-sided 95% CI and p value were calculated. Model effect estimation was based on restricted maximum likelihood (REML). The primary MMRM assumed missing at random (MAR) as a means of handling missing data. Sensitivity analyses of these primary analyses consisted of ANCOVA at month 6 with missing data imputed using five different methods. The results from each of the sensitivity analyses were compared to the primary MMRM analysis.

The analysis of the key secondary efficacy measure, CGI-C at 6 months, was performed using logistic regression, with age at two levels (6-9 years, ≥10 years), sex, treatment, region, visit, and treatment-by-visit interaction as fixed effects. Rank score ANCOVA methodology was used. The responder analysis compared the proportion of participants with improvements (scores 1-3), to those with stable or worsening disease (scores 4-7) between treated groups and the placebo. Results are presented as percentages of participants and logistic analysis odds ratio (OR) with 95% CI and a p-value.

Safety analysis was performed for the Safety Population with no statistical comparisons between the groups. Adverse events were monitored throughout the study and were coded using MedDRA version 24·0. Coding included system organ class and preferred term. All verbatim descriptions and coded terms were listed for all AEs. AEs of special interest, including those which were potentially steroid related and those indicative of adrenal insufficiency, were summarized.

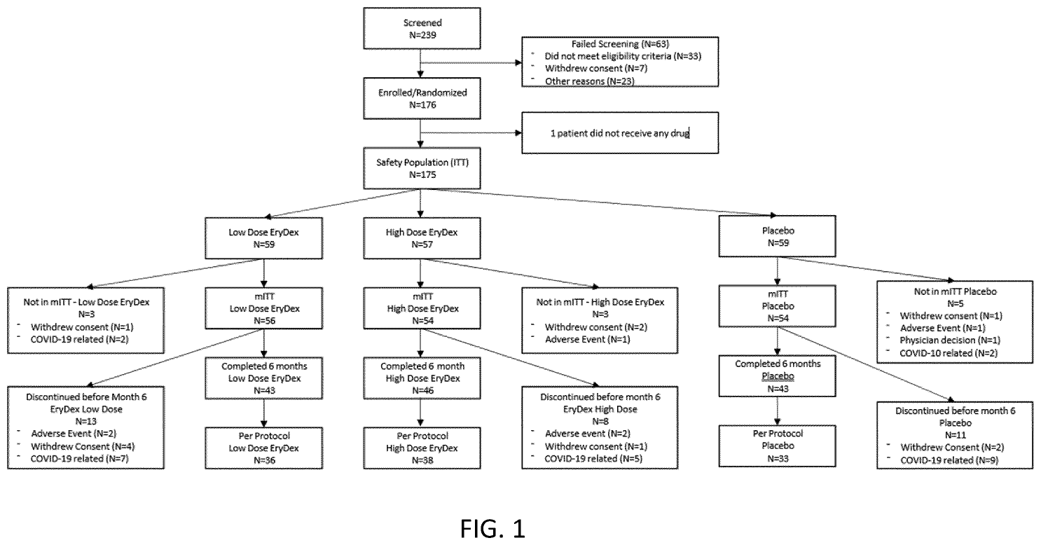

Results. The ATTeST study was conducted in 22 academic institutions and medical centers, situated in 12 different countries on five continents, between Mar. 2, 2017, and May 13, 2021. Out of 239 screened patients, 176 were randomized, but one did not receive study drug. Out of 175 participants who received study drug (intention to treat/safety population), 59 received LD EryDex, 57 HD, and 59 placebo. The mITT population included 164 (93.2%) participants who received at least one dose of drug and had at least one primary efficacy assessment. A total of 132 participants completed the 6-month treatment period for the primary efficacy analysis (43 [72·9%], 46 [80·7%] and 43 [72·9%] for the LD, HD, and placebo group, respectively). The most common reasons for discontinuation before month 6 in LD, HD, and placebo group, respectively, were related to COVID-19 disruptions of treatment (9 [15·3%], 5 [8·8%], and 11 [18·6%]) and withdrawal by subject (5 [8·5%], 3 [5·3%], and 3 [5·1%]). One hundred and seven participants, or 61.1% of the Safety Population, completed treatment as per protocol (PP). FIG. 1 summarizes disposition of participants including rationale for discontinuations from the study. Demographics and baseline neurological and clinical characteristics of all participants who received at least one dose of study drug are presented in Table 1. The randomization process ensured that the groups were balanced by age (6-9 years and≥10 years) and sex. Distribution of all other characteristics and laboratory values were the result of random assignments to treatment groups.

TABLE 1
Patient characteristics by treatment group

| | | |
|---|---|---|
| EryDex Low Dose | EryDex High Dose | Placebo |
| (n = 59) | (n = 57) | (n = 59) |

Age at Randomization/Enrollment [years]

| | | | |
|---|---|---|---|
| Mean (SD) | 9.5 (3.16) | 10.2 (4.86) | 10.3 (4.02) |
| Median (min, max) | 9.0 (6, 19) | 9.0 (6, 37) | 9.0 (6, 29) |
| 6-9 years (%) | 34 (57.6) | 32 (56.1) | 32 (54.2) |
| ≥10 years (%) | 25 (42.4) | 25 (43.9) | 27 (45.8) |

Sex [n (%)]

| | | | |
|---|---|---|---|
| Male | 30 (50.8) | 30 (52.6) | 30 (50.8) |
| Female | 29 (49.2) | 27 (47.4) | 29 (49.2) |

Ethnicity [n (%)

| | | | |
|---|---|---|---|
| Hispanic or Latino | 3 (5.1) | 2 (3.5) | 1 (1.7) |
| Not Hispanic or Latino | 56 (94.9) | 55 (96.5) | 58 (8.3) |

Race [n (%)]

| | | | |
|---|---|---|---|
| White | 34 (57.6) | 36 (63.2) | 29 (49.2) |
| Black or African | 0 | 1 (1.8) | 2 (3.4) |
| American/European | | | |
| Asian | 25 (42.4) | 20 (35.1) | 27 (45.8) |
| Multiple | 0 | 0 | 1 (1.7) |

Baseline modified ICARS Total Score

| | | | |
|---|---|---|---|
| Mean (SD) | 27.9 (7.33) | 27.5 (7.13) | 28.2 (6.50) |
| Median (min, max) | 28.0 (8, 40) | 27.0 (7, 42) | 30.0 (15, 40) |

Alpha Fetoprotein [IU/mL]

| | | | |
|---|---|---|---|
| n | 58 | 55 | 58 |
| Mean (SD) | 277.09 (179.688) | 230.07 (198.721) | 324.18 (187.335) |
| Median (min, max) | 247.20 (2.5, 831.8) | 204.00 (4.0, 1192.0) | 257.10 (8.4, 764.3) |

CD4/Lymphocytes [/μL]

| | | | |
|---|---|---|---|
| Mean (SD) | 489.5 (273.90) | 498.1 (277.81) | 422.9 (223.02) |
| Median (min, max) | 405.0 (136, 1177) | 411.0 (149, 1561) | 371.0 (99, 1307) |

Weight [kg]

| | | | |
|---|---|---|---|
| Mean (SD) | 27.49 (11.018) | 26.92 (12.843) | 27.79 (11.232) |
| Median (min, max) | 25.00 (15.1, 69.8) | 22.63 (15.2, 79.8) | 25.20 (15.1, 73.5) |

Genetic Diagnosis [n (%)]

| | | | |
|---|---|---|---|
| Ataxia Telangiectasia confirmed | 54 (91.5) | 54 (94.7) | 54 (91.5) |

Abbreviations: min = minimum; max = maximum; SD = standard deviation.

Primary efficacy endpoint results, change in mICARS from baseline to month 6 in mITT and PP populations, as assessed by central review based on videotaped exams, for the entire cohort and by age groups are summarized in Table 2. In the mITT population, there was no statistical difference in the primary outcome measures (mICARS) with LSM of −1·37 (p=0·0847) and −1·40 (p=0·0765) in the EryDex LD and HD groups compared to placebo, respectively. However, in participants who received their therapy as per protocol, the effect of treatment was significant in both LD and HD groups with mICARs LSM of −2·80 (p=0·0037) and −2·21 (p=0·0193). In analysis by age, for the mITT population, the treatment effect was significant in 6 to 9-year-olds in the EryDex HD group (LSM −2·79, p=0·0185), but not in the LD group (LSM −1·35, p=0·2392) or in≥10 year olds, regardless of dose (LD: LSM −1·56, p=0·1539 and HD: LSM −0·24, p=0·8158). In the per protocol treated population, the effect of treatment was significant in 6 to 9-year-olds in both LD and HD groups (LSM −3·59, p=0·0071 and −4·39, p=0·0016, respectively), but not in≥10 year olds. Table 5 describes outcomes as measured by ICARS.

TABLE 2
Change in mICARS from baseline to month 6 compared to placebo in all patients and by age

| mITT Population | | | |
|---|---|---|---|
| | EryDex | EryDex | |
| | Low Dose | High Dose | Placebo |
| | (N = 56) | (N = 54) | (N = 54) |

| | | | | | |
|---|---|---|---|---|---|
| Mean Baseline (SD) | 28.0 | (7.38) | 27.5 | (7.29) | 28.4 (6.53) |
| LSM diff. from | −1.37 | (0.0847) | −1.40 | (0.0765) | — |
| placebo (p-value) | | | | | |

| Per Protocol Population | | | |
|---|---|---|---|
| | EryDex | EryDex | |
| | Low Dose | High Dose | Placebo |
| | (N = 36) | (N = 38) | (N = 33) |

| | | | | | |
|---|---|---|---|---|---|
| Mean Baseline (SD) | 27.3 | (8.59) | 27.6 | (6.98) | 28.2 (7.05) |
| LSM diff. from | −2.80 | (0.0037) | −2.21 | (0.0193) | — |
| placebo (p-value) | | | | | |

| | mITT Population | | | | | | | | | |
|---|---|---|---|---|---|---|---|---|---|---|
| | 6-9 Year Olds | | | | | ≥10 Year Olds | | | | |
| | EryDex | | EryDex | | | EryDex | | EryDex | | |
| | Low Dose | | High Dose | | Placebo | Low Dose | | High Dose | | Placebo |
| | (N = 31) | | (N = 29) | | (N = 29) | (N = 25) | | (N = 25) | | (N = 25) |
| Mean baseline (SD) | 25.1 | (7.45) | 27.1 | (6.41) | 26.4 (7.45) | 31.6 | (5.52) | 28.0 | (8.31) | 30.6 (4.41) |
| LSM diff. from | −1.35 | (0.2392) | −2.79 | (0.0185) | — | −1.56 | (0.1539) | −0.24 | (0.8158) | — |
| placebo (p-value) | | | | | | | | | | |
| | Per Protocol Population | | | | | | | | | |
| | 6-9 Year Olds | | | | | ≥10 Year Olds | | | | |
| | EryDex | | Ery Dex | | | EryDex | | EryDex | | |
| | Low Dose | | High Dose | | Placebo | Low Dose | | High Dose | | Placebo |
| | (N = 22) | | (N = 19) | | (N = 19) | (N = 14) | | (N = 19) | | (N = 19) |
| Mean baseline (SD) | 24.0 | (8.23) | 27.6 | (4.28) | 25.3 (7.79) | 32.4 | (6.48) | 27.6 | (9.05) | 32.2 (2.89) |
| LSM diff. from | −3.59 | (0.0071) | −4.39 | (0.0016) | — | −1.71 | (0.2201) | −0.03 | (0.9798) | — |
| placebo (p-value) | | | | | | | | | | |

Abbreviations: Diff = difference; LSM = least squares mean; mICARS = modified International Cooperative Ataxia Rating Scale, mITT = modified intention to treat population; SD = standard deviation.

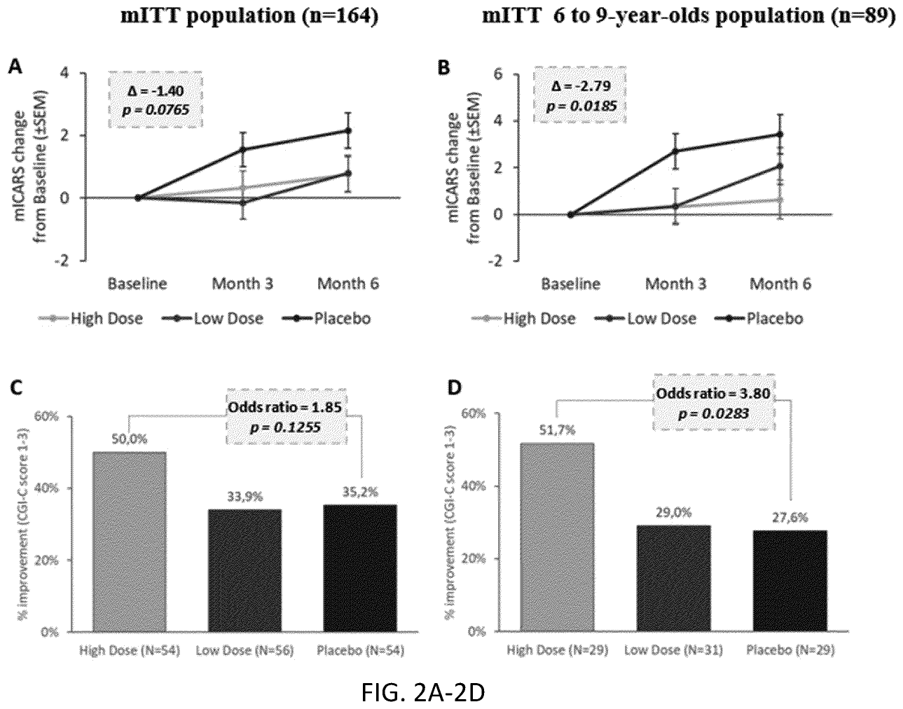

In the key secondary endpoint analysis of disease severity change from baseline to month 6 (CGI-C score), improvement was noted in 19/56 (33·9%), 27/54 (50·0%) and 19/54 (35·2%) of participants in LD, HD, and placebo groups, respectively. Logistic regression OR for LD and HD groups compared to placebo was 0·946 (p=0·8919) and 1·848 (p=0·1255). Stable disease (CGI-C score 4) was documented in 23/56 (41.1%), 17/54 (31.5%) and 22/54 (40.7%) of participants in the LD, HD, and placebo group, respectively. Analysis by age identified that 6 to 9 year-olds receiving HD EryDex had significantly improved CGI-C scores compared to placebo 15/29 (51.7%) vs 8/29 (27.6%) (OR 3·800, p=0·0283), but≥10-year-olds did not (OR 1·208, p=0·7450). Description of CGI-C scores at month 6 is described in Table 6. Summary of main efficacy results are depicted in FIG. 2.

Table 3 depicts a summary of adverse events, by assigned treatment, in the Safety Population. For the entire study period TEAEs were documented in the majority of participants in each treatment group (76-88%), while serious TEAEs were seen in 14-21%. Distribution of TEAEs and serious TEAEs were similar across treatment assignments. Adverse events potentially related to steroid treatment were noted in 30 (51%), 36 (63%), and 23 (39%), of participants in LD, HD, and placebo group during the first 6-month treatment period and in 32 (54%), 42 (74%), and 12 (63%) LD, HD, and non-switched placebo group for the entire period. Potentially steroid-related AEs that were seen more frequently (≥5%) in participants treated with EryDex compared with placebo included influenza, nasopharyngitis, upper respiratory tract infection, weight increase, osteoporosis, and pruritus. Osteopenia was reported in one LD and two HD-treated participants, and osteoporosis in four (7%) patients treated with HD EryDex. There were no reports of hyperglycemia, hypertension, hirsutism, or cushingoid appearance in any of the treatment groups. Hypotension was reported in one participant on HD EryDex and one in the placebo group. Low cortisol levels, without signs of adrenal insufficiency, were reported in one participant from the HD group on day 217. Participants in all three groups started treatment with normal values of hemoglobin A1c and continued to have normal values at month 12. The Columbia-Suicide Severity Scale (C-SSRS) showed no instances of suicidal ideation or behavior at the 6-month evaluation. Irritability was the most common psychiatric complaint, reported in three (5%) LD, one (2%) HD and one (5%) non switched placebo participants. There were no cases of steroid-induced psychosis. One participant treated in LD group developed B cell lymphoma. There were no study-related deaths.

TABLE 3
Summary of treatment-emergent adverse events and more frequent
steroid-related adverse events (Safety Population)

| Initial Treatment (6-month) | | | Entire Treatment (12-month) | | |
|---|---|---|---|---|---|
| EryDex | EryDex | | EryDex | EryDex | |
| LD | HD | Placebo | LD | HD | Placebo* |
| (n = 59) | (n = 57) | (n = 59) | (n = 59) | (n = 57) | (n = 19) |

Treatment Emergent AE [n (%)]

| | | | | | | | | | | | | |
|---|---|---|---|---|---|---|---|---|---|---|---|---|
| Any | 43 | (73) | 47 | (82) | 43 | (73) | 45 | (76) | 50 | (88) | 15 | (79) |
| Serious | 6 | (10) | 7 | (12) | 7 | (12) | 8 | (14) | 9 | (16) | 4 | (21) |

| | | | | | | | | | |
|---|---|---|---|---|---|---|---|---|---|
| Leading to Discontinuation | 0 | 2 | (4) | 0 | 1 | (2) | 2 | (4) | 0 |

| | | | | | | |
|---|---|---|---|---|---|---|
| Leading to Death | 0 | 0 | 0 | 0 | 0 | 0 |

Any potentially steroid-related TEAEs and those identified more frequently
(≥5%) in patients treated with EryDex vs. Placebo [n (%)]

| Any potential steroid-related AE | 30 | (51) | 36 | (63) | 23 | (39) | 32 | (54) | 42 | (74) | 12 | (63) |
|---|---|---|---|---|---|---|---|---|---|---|---|---|
| Influenza | 2 | (3) | 3 | (5) | 0 | | 2 | (3) | 4 | (7) | 1 | (5) |
| Nasopharyngitis | 6 | (10) | 7 | (12) | 4 | (7) | 10 | (17) | 11 | (19) | 5 | (26) |
| Upper respiratory tract infections | 3 | (5) | 4 | (7) | 4 | (7) | 8 | (14) | 9 | (16) | 1 | (5) |

| | | | | | | | | | | | |
|---|---|---|---|---|---|---|---|---|---|---|---|
| Weight Increase | 1 | (2) | 3 | (5) | 2 | (3) | 1 | (2) | 3 | (5) | 0 |

| | | | | | | | | |
|---|---|---|---|---|---|---|---|---|
| Osteoporosis | 0 | 0 | 1 | (2) | 0 | 4 | (7) | 0 |

| | | | | | | | | | | |
|---|---|---|---|---|---|---|---|---|---|---|
| Pruritus | 1 | (2) | 9 | (16) | 0 | 3 | (5) | 10 | (18) | 0 |

*Patients who remained on placebo throughout the study.
AE = adverse events,
HD = high dose;
LD = low dose;
TEAE = treatment emergent adverse events

A full listing of TEAEs, serious TEAE and potentially steroid-related TEAEs is included in Tables 7-10.

The results consistently showed diminished response in gross motor function to treatment in 50 children≥10 years of age. This finding is important for two reasons. First, it emphasizes the need to start treatment in children with A-T early. Second, until a better biomarker of response to therapy is identified, randomized therapeutic trials in patients with A-T should be stratified by age and adequately powered within each age group. A major strength of the ATTeST trial is that it has been developed as a collaborative effort between the world's leading academic clinical and basic science A-T investigators and the sponsor. This collaboration, in addition to evaluating a new agent in a large cohort of children with A-T, led to the development of research assessment tools, such as mICARs and CGI-C, which were refined specifically for the A-T population.

The trial also gathered biologic materials which will be used to address questions related to the mechanism of action (MOA) of steroids in this disease. Initial studies of the MOA of betamethasone in A-T patients indicated that patients with the best neurologic response to therapy also had the highest intracellular glutathione levels. (Russo I et al., Eur J Neurol. 2009;16:755-9.) This effect was confirmed by treatment of A-T lymphoblastoid cell lines with dexamethasone and documenting increased levels of glutathione synthesis and nicotinamide adenine dinucleotide phosphate (NADPH) production. (Biagotti S et al., FEBS J. 2016;283:3962-78.) Molecular studies of nine patients treated with EryDex showed the impact of treatment on gene expression signature with more than 500 upregulated genes and partial restoration of many metabolic pathways typically affected by loss of ATM activity. (Menotta M et al., Orphanet J Rare Dis. 2017;12(1):126. doi:10.1186/s13023-017-0669-2.) Another hypothesis of dexamethasone's MOA is based on an observation that dexamethasone induced alternative ATM gene splicing resulting in a functional “miniATM” protein. (Menotta M et al., J Biol Chem. 2012;287:41352-63; Ricci A et al., Cell Mol Life Sci. 2022;79(12):601.Published online 2022 Nov. 23. doi:10.1007/s00018-022-04625-3.)

The descriptive safety data, showing no difference in frequency of TEAE or serious TEAEs between treated groups and placebo, indicate a favorable safety profile of EryDex. Also, side-effects typically seen with steroid use, such as cushingoid features, were not reported in any of our participants. It is possible that EryDex can cause adrenal suppression as low serum cortisol levels were found in 1 out of 57 (1.8%) participants after 7 months of HD treatment, so patients should be monitored for adrenal suppression when stopping treatment and during intercurrent illness.

Many limitations of our study are inherent to studies of patients with A-T, such as lack of a biomarker for evaluation of treatment efficacy, incomplete understanding of the mechanism of neuronal damage and when it becomes irreversible, difficulties in quantifying progression of disease and response to treatment, complexity of assessment tools, and day-to-day variability in symptomatology. Parents reported that neurological symptoms of patients with A-T significantly deteriorated if the treatment was delayed. Assessing neurological symptoms at a delayed visit may underestimate treatment response. Our trial was conducted during the COVID-19 pandemic. Pandemic control guidelines, including travel restrictions, varied among different countries and institutions, and resulted in a number of skipped treatments and delayed doses. In some instances, investigators delayed infusion of EryDex in order to protect immunocompromised A-T participants from coming to the hospital setting in the peak of the pandemic. Delays in treatment and skipped doses were the major contributor to attrition of participants, resulting in 61% of randomized participants receiving treatment as per protocol. As the analyses showed, EryDex was effective when given as per protocol in the entire population, as well as in the 6 to 9-year-old subgroup.

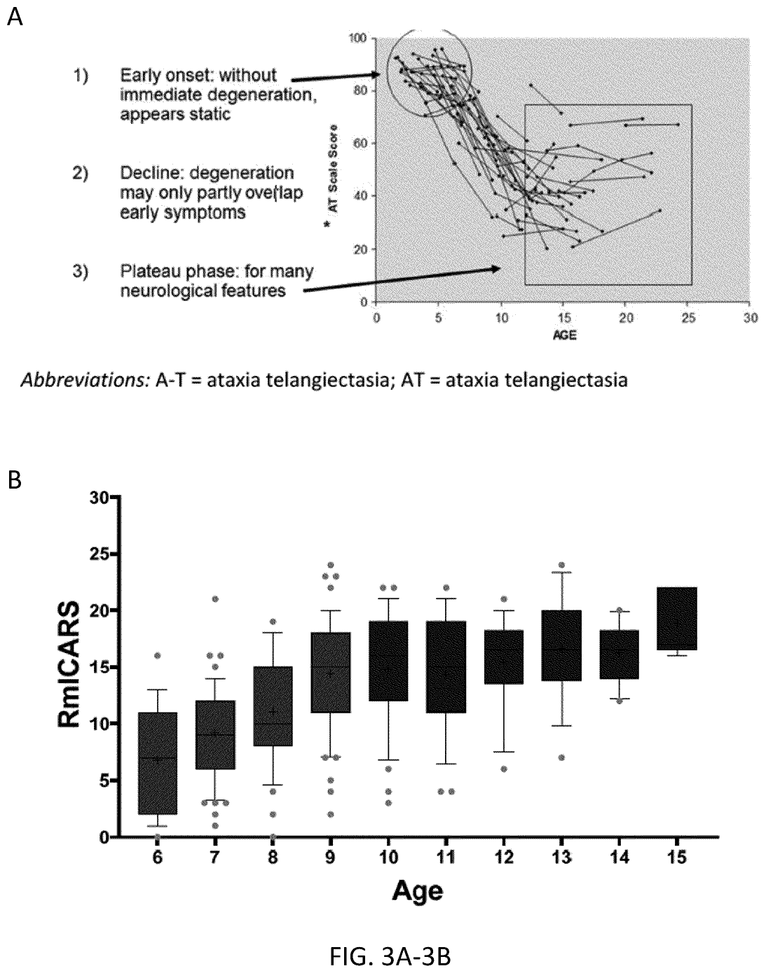

Studies of the natural history of disease progression in patients with classical type of A-T indicated that children<6 years of age have limited clinical signs of neurological deterioration, but between 6 and 10 years of age, there is a rapid clinical decline after which the disease progression stabilizes (Rothblum-Oviatt C et al., Orphanet J Rare Dis 2016;11 (1):159. doi:10.1186/s13023-016-0543-7). This rapid disease progression likely allowed for detection of divergence between treated 6 to 9-year-olds and controls over a short treatment period. Also, treatment of neurodegenerative disorders is more effective earlier in the disease course before significant neuronal damage occurs. As shown in FIG. 3A, subjects<6 years of age show limited clinical signs of neurological deterioration, while between 6 to 10 years of age, there is a rapid clinical deterioration which subsequently plateau.

These natural history data are supported by a cross sectional analysis of the baseline RmICARS scores (an identical pattern can be observed, using Modified International Cooperative Ataxia Rating Scale (mICARS) or full International Cooperative Ataxia Rating Scale (ICARS) scores from a large cohort of A-T subjects (n=261) from the ATTeST study, the Phase 2 IEDAT-ERY01-2010 study and a non-interventional study (Nissenkorn et al., 2015). As shown in FIG. 3B, there are two distinctive regression patterns between subjects 6-9 years and≥10 years of age. There is a rapid clinical manifestation of neurological deterioration of 2.4 RmICARS points/year in the younger age group (6-9 years) as compared to a much slower deterioration of 0.7 RmICARS points/year in older subjects (≥10 years).

In the present study, there was a much larger EryDex treatment effect in the 6-to 9-year-old subpopulation. These findings are aligned with the natural history data that show a slow rate of decline for ataxia symptoms up to age 4-5, increasing from age 6 to 9 and then decreasing significantly from age 10 onwards.

The observed improved safety profile of EryDex compared to oral corticosteroids will need to be confirmed by additional analyses of patients treated with longer-term EryDex. Finally, given the variety of indications for which corticosteroids are used, despite their numerous adverse effects, developing a treatment with maintained or enhanced efficacy and reduced side effects, compared to oral corticosteroids will have significant implications for patients with many other disorders.

TABLE 4
Comparison of ICARS and mICARS, items and scores

| ICARS | | mICARS | |
|---|---|---|---|
| 100 points | | 54 points | |
| 19 Items | | 11 items | |
| Posture and Gait Disturbance (34 points) | | Posture and Gait Disturbance (34 points) | |
| 1. Walking capacities | 0-8 | 1. Walking capacities | 0-8 |
| 2. Gait Speed | 0-4 | 2. Gait Speed | 0-4 |
| 3. Standing Capacities eyes open | 0-6 | 3. Standing Capacities eyes open | 0-6 |
| 4. Spread of feet eyes open | 0-4 | 4. Spread of feet eyes open | 0-4 |
| 5. Body sway feet together eyes open | 0-4 | 5. Body sway feet together eyes open | 0-4 |
| 6. Body sway feet together eyes closed | 0-4 | 6. Body sway feet together eyes closed | 0-4 |
| 7. Quality of sitting position | 0-4 | 7. Quality of sitting position | 0-4 |
| Kinetic Function (52 points) | | Kinetic Function (12 points) | |
| Test left & right except drawing | | Test left & right except drawing | |
| 8. Knee tibia test (R/L) | 0-4 | NA | NA |
| 9. Action tremor (R/L) | 0-4 | NA | NA |
| 10. Finger to nose test (dysmetria) (R/L) | 0-4 | NA | NA |
| 11. Finger to nose test (intention tremor) (R/L) | 0-4 | NA | NA |
| 12. Finger to finger test (R/L) | 0-4 | NA | NA |
| 13. Pronation supination (R/L) | 0-4 | 13. Pronation supination (R/L) | 0-4 |
| 14. Drawing | 0-4 | 14. Drawing | 0-4 |
| Speech Disorder (8 points) | | Speech Disorder (8 points) | |
| 15. Fluency of speech | 0-4 | 15. Fluency of speech | 0-4 |
| 16. Clarity of speech | 0-4 | 16. Clarity of speech | 0-4 |
| Oculomotor Disorders (6 points) | | NA | |
| 17. Gaze evoked nystagmus | 0-3 | NA | NA |

Abbreviations: Diff = difference; LSM = least squares mean; mICARS = modified International Cooperative Ataxia Rating Scale, L = left; mITT = modified intention to treat population; R = right; SD = standard deviation.

TABLE 5
Change in International Cooperative Ataxia Rating Scale (ICARS) from
baseline to month 6 compared to placebo in all patients and by age

| | mITT Population | | |
|---|---|---|---|
| | EryDex | EryDex | |
| | Low Dose | High Dose | Placebo |
| | (N = 56) | (N = 54) | (N = 54) |
| Mean Baseline (SD) | 49.7 (13.00) | 50.0 (12.73) | 51.1 (11.55) |
| LSM diff. from | −0.71 (0.5937) | −1.84 (0.1629) | NA |
| placebo (p-value) | | | |
| | Per Protocol Population | | |
| | EryDex | EryDex | |
| | Low Dose | High Dose | Placebo |
| | (N = 36) | (N = 38) | (N = 33) |
| Mean Baseline (SD) | 49.1 (15.27) | 50.1 (12.40) | 51.5 (12.26) |
| LSM diff. from | −2.46 (0.1317) | −3.16 (0.0512) | NA |
| placebo (p-value) | | | |

| | mITT Population | | | | | | | |
|---|---|---|---|---|---|---|---|---|
| | 6-9 Year Olds | | | ≥10 Year Olds | | | | |
| | EryDex | EryDex | | EryDex | | EryDex | | |
| | Low Dose | High Dose | Placebo | Low Dose | | High Dose | | Placebo |
| | (N = 31) | (N = 29) | (N = 29) | (N = 25) | | (N = 25) | | (N = 25) |
| Mean baseline (SD) | 44.9 (13.58) | 50.5 (10.60) | 48.0 (13.02) | 55.6 | (9.54) | 49.4 | (15.05) | 54.8 (3.44) |
| LSM diff. from | −0.17 (0.9309) | −4.55 (0.0236) | NA | −1.25 | (0.4641) | 0.41 | (0.8037) | NA |
| placebo (p-value) | | | | | | | | |

| | Per Protocol Population | | | | | | | |
|---|---|---|---|---|---|---|---|---|
| | 6-9 Year Olds | | | | ≥10 Year Olds | | | |
| | EryDex | EryDex | | | Ery Dex | EryDex | | |
| | Low Dose | High Dose | | Placebo | Low Dose | High Dose | | Placebo |
| | (N = 22) | (N = 19) | | (N = 19) | (N = 14) | (N = 19) | | (N = 14) |
| Mean baseline (SD) | 44.1 (15.93) | 51.4 | (6.58) | 47.0 (13.83) | 56.9 (10.48) | 48.8 | (16.42) | 57.5 (6.07) |
| LSM diff. from | −3.35 (0.1404) | −6.81 | (0.0046) | NA | −0.55 (0.8034) | 0.79 | (0.7230) | NA |
| placebo (p-value) | | | | | | | | |

Diff. difference;
LSM = least square mean;
mITT = modified intention to treat population;
NA = not applicable;
SDS = standard deviation

TABLE 6
CGI-C scores at month 6, all patients by treatment group and age (mITT)

| EryDex Low Dose | EryDex High Dose | Placebo |
|---|---|---|
| (n = 56) | (1 = 54) | (n = 54) |

CGI-C Score [n (%)]

| | | | |
|---|---|---|---|
| Improvement (Scores 1-3) [n (%)] | 19 (33.9) | 27 (50.0) | 19 (35.2) |
| Stable (Score 4) [n (%)] | 23 (41.1) | 17 (31.5) | 22 (40.7) |
| Worsening (Scores 5-7) [n (%)] | 14 (25.0) | 10 (18.5) | 13 (24.1) |

Logistic Analysis

| | | | |
|---|---|---|---|
| Odds Ratio* (EryDex versus | 0.946 | 1.848 | NA |
| placebo) | | | |
| 95% CI | 0.426-2.099 | 0.842-4.053 | NA |
| p-value | 0.8919 | 0.1255 | NA |

| EryDex Low Dose | | EryDex High Dose | | Placbo | |
|---|---|---|---|---|---|
| 6-9 years | ≥10 years | 6-9 years | ≥10 years | 6-9 years | ≥10 years |
| (N = 31) | (N = 25) | (N = 29) | (N = 25) | (N = 29) | (N = 25) |

CGI-C Score [n (95%)]

| | | | | | | | | | | | | |
|---|---|---|---|---|---|---|---|---|---|---|---|---|
| Improvement (Scores 1-3) [n (%)] | 9 | (29.0) | 10 | (40.0) | 15 | (51.7) | 12 | (48.0) | 8 | (27.6) | 11 | (44.0) |
| Stable (Score 4) [n (%)] | 12 | (38.7) | 11 | (44.0) | 8 | (27.6) | 9 | (36.0) | 13 | (44.8) | 9 | (36.0) |
| Worsening (Scores 5-7) [n (%)] | 10 | (32.3) | 4 | (16.0) | 6 | (20.7) | 4 | (16.0) | 8 | (27.6) | 5 | (20.0) |

Logistic Analysis

| Odds Ratio* (EryDex versus | 1.333 | 0.922 | 3.800 | 1.208 | NA | NA |
|---|---|---|---|---|---|---|
| placebo) | | | | | | |
| 95% CI | 0.404-4.396 | 0.292-2.911 | 1.152-12.530 | 0.387-3.772 | NA | NA |
| p-value | 0.6373 | 0.8903 | 0.0283 | 0.7450 | NA | NA |

Abbreviations: CGI-C = Clinical Global Impression of Change.
*Odds ratio analysis compared participants (EryDex vs placebo) with improved score vs. those with stable/worsening of CGI-C score

TABLE 7
Overall summary of adverse events in safety population

| | 6-Month Initial Treatment Period | | | | | | Entire Treatment Period | | | | | |
|---|---|---|---|---|---|---|---|---|---|---|---|---|
| | EryDex | | EryDex | | | | EryDex | | EryDex | | Non-switch | |
| | Low Dose | | High Dose | | Placebo | | Low Dose | | High Dose | | Placebo | |
| | (N = 59) | | (N = 47) | | (N = 59) | | (N = 59) | | (N = 57) | | (N = 19) | |
| Patients With Pre-treatment AE | 14 | (23.7) | 16 | (28.1) | 14 | (23.7) | 14 | (23.7) | 16 | (28.1) | 6 | (31.6) |
| [n (%)] | | | | | | | | | | | | |
| Patients With Any TEAE [n (%)] | 43 | (72.9) | 47 | (82.5) | 43 | (72.9) | 45 | (76.3) | 50 | (87.7) | 15 | (78.9) |
| Total Number of TEAEs | 202 | | 251 | | 155 | | 326 | | 454 | | 103 | |
| Patients With Any Treatment- | 15 | (25.4) | 21 | (36.8) | 15 | (25.4) | 19 | (32.2) | 25 | (43.9) | 5 | (26.3) |
| related TEAE [n (%)] | | | | | | | | | | | | |
| Patients With Any Serious TEAE | 6 | (10.2) | 7 | (12.3) | 7 | (11.9) | 8 | (13.6) | 9 | (15.8) | 4 | (21.1) |
| [n (%)] | | | | | | | | | | | | |

| | | | | | | | | | | |
|---|---|---|---|---|---|---|---|---|---|---|
| Patients With Any Serious | 0 | 1 | (1.8) | 0 | 1 | (1.7) | 1 | (1.8) | 1 | (5.3) |

Treatment-related TEAE [n (%)]

| | | | | | | | | | |
|---|---|---|---|---|---|---|---|---|---|
| Patients With Any TEAE Leading | 0 | 2 | (3.5) | 0 | 1 | (1.7) | 2 | (3.5) | 0 |

| to Discontinuation [n (%)] | | | | | | |
|---|---|---|---|---|---|---|
| Patients With Any TEAE Leading | 0 | 0 | 0 | 0 | 0 | 0 |
| to Death [n (%)] | | | | | | |

Abbreviations: AE = adverse event; TEAE = treatment-emergent adverse events.

TABLE 8
Most commonly occurring TEAEs (≥2 Patients by MedDRA preferred term) in safety population

| | 6 Month Initial Treatment Period | | | | | Entire Treatment Period | | | | |
|---|---|---|---|---|---|---|---|---|---|---|
| | EryDex | | EryDex | | | EryDex | EryDex | | Non-switch | |
| System Organ Class [n (%)] | Low Dose | | High Dose | | Placebo | Low Dose | High Dose | | Placebo | |
| Preferred Term [n (%)] | (N = 59) | | (N = 57) | | (N = 59) | (N = 59) | (N = 57) | | (N = 19) | |
| Any Related TEAE [n (%)] | 15 | (25.4) | 21 | (36.8) | 15 (25.4) | 19 (32.2) | 25 | (43.9) | 5 | (26.3) |
| Blood and Lymphatic System | 1 | (17) | 1 | (1.8) | 2 (3.4) | 1 (1.7) | 2 | (3.5) | 0 | |
| Disorders | | | | | | | | | | |
| Anaemia | 0 | | 1 | (1.8) | 1 (1.7) | 0 | 1 | (1.8) | 0 | |
| Gastrointestinal Disorders | 3 | (5.1) | 4 | (7.0) | 3 (5.1) | 4 (6.8) | 5 | (3.8) | 0 | |
| Abdominal Pain | 0 | | 1 | (1.8) | 0 | 0 | 2 | (3.5) | 0 | |
| Abdominal Pain Upper | 0 | | 1 | (1.8) | 0 | 0 | 1 | (1.8) | 0 | |
| Anal Pruritus | 0 | | 2 | (3.5) | 0 | 0 | 2 | (3.5) | 0 | |
| Diarrhea | 1 | (1.7) | 0 | | 1 (1.7) | 1 (1.7) | 0 | | 0 | |
| Nausea | 3 | (5.1) | 0 | | 1 (1.7) | 3 (5.1) | 1 | (1.8) | 0 | |
| Vomiting | 3 | (5.1) | 0 | | 0 | 4 (6.8) | 0 | | 0 | |
| General Disorders and | 1 | (1.7) | 5 | (8.8) | 3 (5.1) | 3 (5.1) | 9 | (15.8) | 2 | (10.5) |
| Administration Site Conditions | | | | | | | | | | |
| Fatigue | 1 | (1.7) | 2 | (3.5) | 1 (1.7) | 2 (3.4) | 2 | (3.5) | 0 | |
| Pain | 0 | | 1 | (1.8) | 0 | 1 (1.7) | 2 | (3.5) | 0 | |
| Pyrexia | 1 | (1.7) | 1 | (1.8) | 1 (1.7) | 1 (1.7) | 2 | (3.5) | 1 | (5.3) |
| Investigations | 6 | (10.2) | 10 | (17.5) | 3 (5.1) | 8 (13.6) | 12 | (21.1) | 1 | (5.3) |
| Bacterial Test Positive | 1 | (1.7) | 0 | | 0 | 2 (3.4) | 0 | | 1 | (5.3) |
| Neutrophil Count Decreased | 0 | | 1 | (1.8) | 0 | 0 | 2 | (3.5) | 0 | |
| Weight Increased | 1 | (1.7) | 3 | (5.3) | 2 (3.4) | 1 (1.7) | 3 | (5.3) | 0 | |
| White Blood Cell Count | 1 | (1.7) | 2 | (3.5) | 0 | 1 (1.7) | 2 | (3.5) | 0 | |
| Increased | | | | | | | | | | |
| Metabolism and Nutrition | 4 | (6.8) | 2 | (3.5) | 2 (3.4) | 5 (8.5) | 3 | (5.3) | 1 | (5.3) |
| Disorders | | | | | | | | | | |
| Increased Appetite | 2 | (3.4) | 0 | | 2 (3.4) | 2 (3.4) | 1 | (1.8) | 1 | (5.3) |
| Iron Deficiency | 3 | (5.1) | 1 | (1.8) | 0 | 4 (6.8) | 1 | (1.8) | 0 | |
| Nervous System Disorders | 4 | (6.8) | 3 | (5.3) | 2 (3.4) | 5 (8.5) | 5 | (5.5) | 1 | (5.3) |
| Dizziness | 0 | | 0 | | 0 | 0 | 2 | (3.5) | 0 | |
| Headache | 4 | (6.8) | 2 | (3.5) | 0 | 5 (8.5) | 2 | (3.5) | 0 | |
| Psychiatric Disorders | 2 | (3.4) | 2 | (3.5) | 3 (5.1) | 2 (3.4) | 2 | (3.5) | 1 | (5.3) |
| Irritability | 2 | (3.4) | 1 | (1.8) | 1 (1.7) | 2 (3.4) | 1 | (1.8) | 1 | (5.3) |
| Respiratory, Thoracic and | 0 | | 1 | (1.8) | 2 (3.4) | 0 | 3 | (5.3) | 1 | (5.3) |
| Mediastinal Disorders | | | | | | | | | | |

| | | | | | | | | |
|---|---|---|---|---|---|---|---|---|
| Rhinorrhea | 0 | 0 | 1 (1.7) | 0 | 2 | (3.5) | 1 | (5.3) |

| | | | | | | | | | |
|---|---|---|---|---|---|---|---|---|---|
| Skin and Subcutaneous Tissue | 1 | (1.7) | 9 | (15.8) | 2 (3.4) | 3 (5.1) | 10 | (17.5) | 0 |
| Disorders | | | | | | | | | |

| | | | | | | | | |
|---|---|---|---|---|---|---|---|---|
| Pruritus | 0 | 9 | (15.8) | 0 | 2 (3.4) | 10 | (17.5) | 0 |

Abbreviations: MedDRA = Medical Dictionary for Regulatory Activities; TEAE = treatment-emergent adverse event.

TABLE 9
Serious TEAEs by MedDRA preferred term (safety population)

| | 6-Month Initial Treatment Period | | | | | | Entire Treatment Period | | | | | |
|---|---|---|---|---|---|---|---|---|---|---|---|---|
| | EryDex | | Ery Dex | | | | EryDex | | Ery Dex | | Non-switch | |
| System Organ Class [n (%)] | Low Dose | | High Dose | | Placebo | | Low Dose | | High Dose | | placebo | |
| Preferred Term [n (%)] | (N = 59) | | (N = 57) | | (N = 59) | | (N = 59) | | (N = 57) | | (N = 19) | |
| Any serious TEAE [n (%)] | 6 | (10.2) | 7 | (12.3) | 7 | (11.9) | 5 | (13.6) | 9 | (15.8) | 4 | (21.1) |
| Blood and Lymphatic System | 0 | | 1 | (1.8) | 0 | | 0 | | 1 | (1.9) | 0 | |
| Disorders | | | | | | | | | | | | |
| Anemia | 0 | | 1 | (1.8) | 0 | | 0 | | 1 | (1.8) | 0 | |
| Congenital, Familial and Genetic | 0 | | 0 | | 1 | (1.7) | 0 | | 0 | | 0 | |
| Disorders | | | | | | | | | | | | |
| Hepato-Lenticular Degeneration | 0 | | 0 | | 1 | (1.7) | 0 | | 0 | | 0 | |
| General Disorders and | 0 | | 0 | | 1 | (1.7) | 0 | | 1 | (1.8) | 0 | |
| Administration Site Conditions | | | | | | | | | | | | |
| Pyrexia | 0 | | 0 | | 1 | (1.7) | 0 | | 1 | (1.8) | 0 | |
| Infections and Infestations | 2 | (3.4) | 1 | (1.8) | 0 | | 2 | (3.4) | 1 | (1.8) | 0 | |
| Herpes Zoster | 1 | (1.7) | 0 | | 0 | | 1 | (1.7) | 0 | | 0 | |
| Lower Respiratory Tract | 1 | (1.7) | 0 | | 0 | | 1 | (1.7) | 0 | | 0 | |
| Infection | | | | | | | | | | | | |
| Pneumonia | 1 | (1.7) | 0 | | 0 | | 1 | (1.7) | 0 | | 0 | |
| Sepsis | 0 | | 1 | (1.8) | 0 | | 0 | | 1 | (1.8) | 0 | |
| Investigations | 3 | (5.1) | 5 | (8.8) | 4 | (6.8) | 4 | (6.8) | 6 | (10.5) | 3 | (15.8) |
| Bacterial Test Positive | 3 | (5.1) | 5 | (8.8) | 4 | (6.8) | 4 | (6.8) | 6 | (10.5) | 3 | (15.8) |

| | | | | | | | | |
|---|---|---|---|---|---|---|---|---|
| Musculoskeletal and Connective | 0 | 0 | 1 | (1.7) | 0 | 0 | 1 | (5.3) |
| Tissue Disorders | | | | | | | | |
| Juvenile Idiopathic Arthritis | 0 | 0 | 1 | (1.7) | 0 | 0 | 1 | (5.3) |

| Neoplasms Benign, Malignant | 0 | | 0 | 0 | 1 | (1.7) | 0 | 0 |
|---|---|---|---|---|---|---|---|---|
| and Unspecified (Incl Cysts and | | | | | | | | |
| Polyps) | | | | | | | | |
| B-Cell Lymphoma | 0 | | 0 | 0 | 1 | (1.7) | 0 | 0 |
| Nervous System Disorders | 1 | (1.7) | 0 | 0 | 1 | (1.7) | 0 | 0 |
| Dystonia | 1 | (1.7) | 0 | 0 | 1 | (1.7) | 0 | 0 |

| | | | | | | | |
|---|---|---|---|---|---|---|---|
| Respiratory, Thoracic and | 0 | 0 | 0 | 1 | (1.7) | 0 | 0 |
| Mediastinal Disorders | | | | | | | |
| Bronchitis Chronic | 0 | 0 | 0 | 1 | (1.7) | 0 | 0 |

| | | | | | | | |
|---|---|---|---|---|---|---|---|
| Vascular Disorders | 0 | 0 | 0 | 0 | 1 | (1.8) | 0 |
| Aortic Stenosis | 0 | 0 | 0 | 0 | 1 | (1.8) | 0 |

Abbreviations: MedDRA = Medical Dictionary for Regulatory Activities; TEAE = treatment-emergent adverse event.

TABLE 10
Potentially steroid-related TEAEs (≥2 patients by preferred term) (safety population)

| | 6-Month Initial Treatment Period | | | | | | Entire Treatment Period | | | | | |
|---|---|---|---|---|---|---|---|---|---|---|---|---|
| | EryDex | | Ery Dex | | | | EryDex | | EryDex | | Non-switch | |
| System Organ Class [n (%)]^{b} | Low Dose | | High Dose | | Placebo | | Low Dose | | High Dose | | Placebo | |
| Preferred Term [n (%)] | (N = 59) | | (N = 57) | | (N = 59) | | (N = 59) | | (N = 57) | | (N = 19) | |
| Any potentially steroid-related | 30 | (50.8) | 36 | (63.2) | 23 | (39.0) | 32 | (54.2) | 42 | (73.7) | 12 | (63.2) |
| TEAE [n (%)] | | | | | | | | | | | | |
| Blood and Lymphatic System | 2 | (3.4) | 1 | (1.8) | 0 | | 2 | (3.4) | 2 | (3.5) | 0 | |
| Disorders | | | | | | | | | | | | |
| Leukopenia | 2 | (3.4) | 0 | | 0 | | 2 | (3.4) | 1 | (1.8) | 0 | |
| Gastrointestinal Disorders | 0 | | 2 | (3.5) | 1 | (1.7) | 0 | | 2 | (3.5) | 0 | |
| Anal Pruritus | 0 | | 2 | (3.5) | 1 | (1.7) | 0 | | 2 | (3.5) | 0 | |
| General Disorders and | 0 | | 1 | (1.8) | 0 | | 0 | | 3 | (5.3) | 0 | |
| Administration Site Conditions | | | | | | | | | | | | |
| Infusion Site Pruritus | 0 | | 1 | (1.8) | 0 | | 0 | | 2 | (3.5) | 0 | |
| Infections and Infestations | 22 | (37.3) | 17 | (29.8) | 13 | (22.0) | 30 | (50.8) | 27 | (47.4) | 8 | (42.1) |
| Bronchitis | 3 | (5.1) | 0 | | 2 | (3.4) | 4 | (6.8) | 2 | (3.5) | 1 | (5.3) |
| Bronchitis Bacterial | 1 | (1.7) | 1 | (1.8) | 0 | | 2 | (3.4) | 1 | (1.8) | 0 | |
| Conjunctivitis | 2 | (3.4) | 1 | (1.8) | 0 | | 2 | (3.4) | 1 | (1.8) | 0 | |
| Herpes Zoster | 1 | (1.7) | 0 | | 0 | | 2 | (3.4) | 0 | | 0 | |
| Influenza | 2 | (3.4) | 3 | (5.3) | 0 | | 2 | (3.4) | 4 | (7.0) | 1 | (5.3) |
| Lower Respiratory Tract | 2 | (3.4) | 1 | (1.8) | 0 | | 2 | (3.4) | 1 | (1.8) | 0 | |
| Infection | | | | | | | | | | | | |
| Nasopharyngitis | 6 | (10.2) | 7 | (12.3) | 4 | (6.8) | 10 | (16.9) | 11 | (19.3) | 5 | (26.3) |
| Otitis Media | 0 | | 2 | (3.5) | 0 | | 1 | (1.7) | 2 | (3.5) | 0 | |
| Respiratory Tract Infection | 1 | (1.7) | 0 | | 1 | (1.7) | 2 | (3.4) | 0 | | 1 | (5.3) |
| Sinusitis | 1 | (1.7) | 0 | | 0 | | 2 | (34) | 1 | (1.8) | 0 | |
| Upper Respiratory Tract | 3 | (5.1) | 4 | (7.0) | 4 | (6.8) | 8 | (13.6) | 9 | (15.8) | 1 | (5.3) |
| Infection | | | | | | | | | | | | |
| Investigations | 4 | (6.8) | 7 | (12.3) | 3 | (5.1) | 6 | (10.2) | 10 | (17.5) | 1 | (5.3) |
| Blood Triglycerides Increased | 0 | | 2 | (3.5) | 1 | (1.7) | 1 | (1.7) | 2 | (3.5) | 1 | (5.3) |
| Bone Density Decreased | 1 | (1.7) | 1 | (1.8) | 0 | | 1 | (1.7) | 2 | (3.5) | 0 | |
| Neutrophil Count Decreased | 0 | | 1 | (1.8) | 0 | | 0 | | 2 | (3.5) | 0 | |
| Weight Increased | 1 | (1.7) | 3 | (5.3) | 2 | (3.4) | 1 | (1.7) | 3 | (5.3) | 0 | |
| Metabolism and Nutrition | 3 | (5.1) | 2 | (3.5) | 2 | (3.4) | 3 | (5.1) | 3 | (5.3) | 1 | (5.3) |
| Disorders | | | | | | | | | | | | |
| Hypertriglyceridemia | 0 | | 2 | (3.5) | 0 | | 0 | | 2 | (3.5) | 0 | |
| Increased Appetite | 2 | (3.4) | 0 | | 2 | (3.4) | 2 | (3.4) | 1 | (1.8) | 1 | (5.3) |
| Musculoskeletal and Connective | 0 | | 0 | | 1 | (1.7) | 1 | (1.7) | 6 | (10.5) | 0 | |
| Tissue Disorders | | | | | | | | | | | | |
| Osteopenia | 0 | | 0 | | 0 | | 1 | (1.7) | 2 | (3.5) | 0 | |
| Osteoporosis | 0 | | 0 | | 1 | (1.7) | 0 | | 4 | (7.0) | 0 | |
| Neoplasms Benign, Malignant | 0 | | 1 | (1.8) | 1 | (1.7) | 1 | (1.7) | 3 | (5.3) | 1 | (5.3) |
| and Unspecified (Incl Cysts and | | | | | | | | | | | | |
| Polyps) | | | | | | | | | | | | |
| Skin Papilloma | 0 | | 1 | (1.8) | 1 | (1.7) | 1 | (1.7) | 3 | (5.3) | 1 | (5.3) |
| Respiratory, Thoracic and | 8 | (13.6) | 10 | (17.5) | 8 | (13.6) | 11 | (18.6) | 14 | (24.6) | 6 | (31.6) |
| Mediastinal Disorders | | | | | | | | | | | | |
| Cough | 7 | (11.9) | 9 | (15.8) | 8 | (13.6) | 10 | (16.9) | 13 | (22.8) | 6 | (31.6) |
| Productive Cough | 1 | (1.7) | 1 | (1.8) | 0 | | 2 | (3.4) | 2 | (3.5) | 0 | |
| Skin and Subcutaneous Tissue | 1 | (1.7) | 9 | (15.8) | 1 | (1.7) | 3 | (5.1) | 10 | (17.5) | 1 | (5.3) |
| Disorders | | | | | | | | | | | | |

| | | | | | | | | | | |
|---|---|---|---|---|---|---|---|---|---|---|
| Pruritus | 1 | (1.7) | 9 | (15.8) | 0 | 3 | (5.1) | 10 | (17.5) | 0 |

Abbreviations: MedDRA = Medical Dictionary for Regulatory Activities; TEAE = treatment-emergent adverse event.

### Additional Material and Methods

### Process Solutions:

Hypotonic Solution 1: each 400 mL bag contains 400 mL saline solution (NaCl 0.563%) in Water for Injection at pH of 5.75 and a target osmolality of 180 mOsm/kg. The solution is sterile and non-pyrogenic. Each bag is packaged with an aluminum over-pouch and stored at ambient temperature.

Hypotonic Solution 2: each 200 mL bag contains 200 mL saline solution (NaCl 0.372%) in Water for Injection at pH of 5.75 and a target osmolality of 120 mOsm/kg. The solution is sterile and non-pyrogenic. Each bag is packaged with an aluminum over-pouch and stored at ambient temperature.

PIGPA Hypertonic Solution: each 5mL vial contains the ingredients listed below in Table 11. Each single use sterile and non-pyrogenic vial is formulated at pH 7.4 and a target osmolality of 3785 mOsm/kg. Each vial is packaged with an aluminum over-pouch and is stored between 2 to 8° C.

TABLE 11
Composition of PIGPA Hypertonic Solution

| Component | Concentration (mg/mL) |
|---|---|
| NaH₂PO4•H₂O | 4.6 |
| KCl | 124 |
| NaCl | 11 |
| Inosine | 27 |
| Adenosine triphosphate | 11 |
| Glucose Anhydrous | 18 |
| Sodium Pyruvate | 11 |
| MgCl₂•6H₂O | 0.8 |
| Water for Injection | q.s. |

The salinity and volume of each Hypotonic Solution used during the EDS process have been studied to gradually reduce the osmotic condition of RBCs suitable to enable encapsulation of the DSP by passive diffusion.

The Hypertonic Solution PIGPA is formulated at high concentration of potassium and sodium chloride suitable to restore physiological osmolality. The “non-salt” components of PIGPA, which are normally present at high concentration into the RBCs, contribute to further increase the osmolarity and to limit the diffusion of the native RBCs components out of the cells during the restoration phase.

Dexamethasone Sodium Phosphate Solution 25 mg/mL. Dexamethasone sodium phosphate, a synthetic corticosteroid, is formulated at 25 mg/mL in water for injection to produce the drug product, DSP Solution. In the EDS, DSP acts as a pro-drug of dexamethasone, a synthetic corticosteroid widely used and long available as a sterile injectable solution dosage form for systemic administration. DSP Solution is sterile and non-pyrogenic, provided separately in single use containing 250 mg DSP in 10 mL water for injection. The drug product is intended to be used ONLY in conjunction with performing the EDS process. The DSP solution is stored between 2 to 8° C.

The EryDex drug product is prepared using 5.0 mL of the 25 mg/mL DSP solution, plus 11 mL sterile water for injection in the same syringe, for a total of 16 mL, corresponding to 125 mg of experimental drug which resulted in 17.4±5.38 mg (mean±SD) of DSP loaded into/encapsulated in RBC and re-infused to subjects. The non-encapsulated DSP is automatically washed away during the EDS process by the RCL, before re-infusion into the same subject.

### Example 2: Reduced Initial Blood Volume for Pediatric Subjects With Low Body Weight

A series of studies were conducted to evaluate EryDex System (EDS) performance using a reduced initial blood volume of 30 ml and a dexamethasone sodium phosphate (DSP) loading quantity of 125 mg, aiming at extending the use of the EDS to pediatric patients with body weights as low as 9 kg. A blood withdrawal between 1% and 5% of total blood volume in a single draw (or over 24 hours) is considered safe, making this low blood volume EDS process crucial to enable treatment for younger, smaller patients.

The resulting encapsulated DSP dose using the 30 ml process was 13.4±0.6 mg/bag, with an 11% encapsulation efficiency and low variability (4%). The mean content of DSP per million RBC was 218±16 ng, similar to eDSP produced using 50 ml of blood volume (i.e., 189±8 ng). This demonstrates that the DSP concentration encapsulated within RBC remains consistent. The 30 ml of blood decreased the DSP dose accordingly, however the proportion of DSP encapsulated in the RBC is largely preserved.

Hematological parameters showed RBC count of 0.82±0.05 106/μl, total hemoglobin (HGB) of i.2±0.1 g/dl, and hematocrit (HCT) of 7.4±0.3. Mean corpuscular hemoglobin (MCH) and mean corpuscular hemoglobin concentration (MCHC) were 15±1.1 pg and 16.7±1.1 g/dl, respectively. A series of parameters were assessed and compared to acceptance criteria previously established for the 50 ml blood volume encapsulated DSP (eDSP) process. RBC recovery (46.3±2.5%) met the acceptance criterion of 2:40% and mean corpuscular volume (MCV) variation was within the acceptable range of±10% of the initial MCV blood value. Osmolality, a critical factor in the encapsulation process, was measured across multiple critical EDS phases; all values were within the predefined acceptance ranges established for the standard SO ml blood process.

The eDSP pH was 7.09±0.03, meeting the 7.0±0.2 acceptance range. Free HGB, measured at the end of the final washing phase, was below the limit of detection (0.01 g/dl). Endotoxin levels were also below the limit of detection of the assay (LLOD=0.13 EU/ml), which is significantly below the maximum tolerated threshold of 0.57 EU/ml.

Together, the results of these studies demonstrated that eDSP can be effectively and reproducibly prepared using a reduced initial blood volume of 30 ml without compromising product quality, safety, or process reliability. The EryDex System (EDS) process was successfully adapted for an initial blood volume of 30 ml, which expands the therapeutic applicability of eDSP to pediatric patients weighing as little as 9 kg. The use of a 30 ml blood volume would allow the treatment of children with a body weight between 9 and 15 kg.

Experimental Methods: A total of 8 EDS processes were performed starting from 30 ml of initial blood volume Blood was collected from eight healthy volunteers in bags of 450 ml plus 64 ml of citrate phosphate dextrose (CPD) as an anticoagulant; each bag was used for a single process. The Red Cell Loader (RCL) software was modified to allow for introduction of 30 ml blood volume during phase one. Other than acceptance of 30 ml initial blood volume, no other changes were made to the current EDS software.

Blood volume was adjusted in the Syringe-kit as appropriate to compensate for anticoagulant-induced changes in volume and differences between subjects, and to approximate a hematocrit (HCT) physiological value of 40±1% in all the processes.

A DSP loading quantity was selected (i.e., 125 mg) for all the EDS processes. Drug preparation was performed diluting 5 ml of DSP vial [25 mg/ml] with 11 ml of Water for Injection (WFI) in a 20 ml syringe. In accordance with EDS procedure, 3 ml of PIGPA were added into the system when requested by the automated process.

During each EDS process, the osmolality was measured six times to confirm that the decrease in the volume of blood does not impact the EDS process osmolality. The samples were collected during phases 5, 9, 11, 12, 14 of the EDS process, and in the eDSP. Additional samples of eDSP were collected and immediately used to evaluate CBC) and pH; an aliquot of 1 ml was stored at −20° C. for DSP quantification; and 5 ml were stored at −20° C. for endotoxin quantification.

Blood Volume Preparation. The correct volume of initial whole blood (WB) required for the EDS process was calculated by the following formula:

$$Vi = \frac{HCTt * Vt}{HCTi}$$

where Vi is the initial blood to be collected; HCTi is the initial HCT of the WB; Vt is the target volume (i.e., 30 ml); HCTt is the target HCT (i.e., 40%). In case HCTi is lower than HCTt, the blood volume Vi was directly used. In case the HCTi is higher than HCTt, injectable saline solution was added to the blood volume Vi to reach the target volume of 30 ml.

Hematological parameters in CBC. Erythrocyte Mean cellular volume (MCV), Mean corpuscular hemoglobin (MCH), Mean corpuscular hemoglobin concentration (MCHC), hematocrit (HCT), Red Blood Cells (RBC), White Blood Cells (WBC), Platelets (PLT), and total hemoglobin (HGB) were all measured using an automatic Cell Coulter in 100 μL sample.

### Results:

DSP dose and volume. The mean DSP dose was 13.4±0.6 mg in a final bag volume of 74.7±0.9 ml. In terms of reproducibility of the process, the variability was equal to 4% with 11% encapsulation efficiency. The sample size of eight provided a 100% power to observe a variation of 20% in DSP dose with respect to the selected reference (12.3 mg/bag reported in the Study Report 2019-P04-R04). The actual variation was 8.9%, well within the pre-defined limit. MCD resulted in 218±16 ng/106 RBC. MCD calculated for encapsulated DSP (eDSP) produced using 50 ml of blood and with the same DSP initial quantity was 189±8 ng/10⁶ RBC.

Hematological parameters (CBC). RBC count was 0.82±0.05 10⁶/μL, WBC and PLT count were 0.5±0.2 103/μL and 5.4±2.5 103/μL, respectively. Total HGB value was 1.2±0.1 g/dl, HCT was 7.4±0.3%, MCH and MCHC were 15±1.1 pg and 16.7±1.1 g/dl, respectively (Table 3). RBC recovery at the end of the EDS process was 46.3±2.5%, with the minimum value of 43.5%. This result matches the 40% minimum acceptance limit set for EDS process. The MCV value was 89.7±5.3 fl, with an MCV variation between eDSP and the initial blood MCV of −1.4±2.9% (maximum variation −5.1%), which is also within the±10% acceptance criterion for eDSP. Whole blood parameters did not show any meaningful deviation from normality.

Osmolality. Osmolality drives the encapsulation process and was recorded throughout the entire EDS process. Osmolality was 203±3 mOsm/kg in phase 5, 173±1 mOsm/kg in phase 9, and 43±1 mOsm/kg in drug preparation (phase 11).; During phase 12, osmolality was 123±2 mOsm/kg, and in phase 14 it was 318±10 mOsm/kg. In eDSP, osmolality was 292±6 mOsm/kg. All data recorded were compared with the acceptance criteria and no deviations were found; all data were inside the defined acceptance criteria.

### Discussion and Overall Conclusions

These studies were conducted to evaluate EDS performance using a reduced initial blood volume of 30 ml and a DSP loading quantity of 125 mg to support the use of the EDS in pediatric patients with body weights as low as 9 kg. The mean DSP dose in eDSP was 13.4±0.6 mg in a final volume of 74.7±0.9 ml volume with limited variability. This consistency indicates that the reduction in blood volume does not compromise the reproducibility of eDSP dosage, a critical parameter for treatment efficacy and dosing reproducibility.

The modified process achieved an MCD of 218±16 ng/106 RBC, closely aligned with the MCD value of 189±8 ng/106 RBC calculated for eDSP produced using a larger (SO ml) blood volume. This difference reflects likely blood and process variability, however the DSP concentration encapsulated within RBC remained consistent, supporting the expectation of similar pharmacodynamic activity.

Hematological parameters confirmed that the EDS process with 30 ml of blood produces an eDSP with characteristics proportionate to those obtained by 50 ml blood. Comparing results of this study with the overall mean results from the ATTeST clinical study (Study Report 2022-P02-R02), the RBC count was 37% lower and HGB was 40% lower, which is explained by the 40% lower initial blood volume used in the 30 ml process compared with the 50 ml blood process used in ATTeST. MCH and MCHC are not related to blood quantity and were 3% lower and 1% higher, respectively, showing strong process robustness with lower blood volumes. RBC recovery and MCV variation remained within acceptance criteria, further validating process reproducibility. Osmolality measurements across all process phases were consistent with the validated 50 ml process, confirming that the RBCs were being processed under the same controlled conditions as the 50 ml process, throughout encapsulation steps.

All other parameters were also met: pH values were within the expected range for RBCs resuspended in injectable saline, and free hemoglobin and endotoxin levels were consistently below the limit of detection (LLOD). The endotoxin at the LLOD supports the safety of eDSP and is significantly lower than the endotoxin limits in accordance with Ph Eur and USP. In conclusion, the study demonstrated that using a reduced initial blood volume of 30 ml for the eDSP process met all performance, reproducibility and safety criteria. These results support the treatment of pediatric patients with a body weight as low as 9 kg with eDSP, while maintaining the same conditions developed using the 50 ml EDS process.

### Alternative Embodiments

Embodiment 1. A method at treating Ataxia Telangiectasia in a subject less than about 10 years of age, comprising administering to the subject at least 5 mg of dexamethasone sodium phosphate (DSP) that has been loaded into erythrocytes.

Embodiment 2. A method at treating Ataxia Telangiectasia in a subject, comprising administering to the subject at least 5 mg of dexamethasone sodium phosphate (DSP) that has been loaded into erythrocytes.

Embodiment 3. The method of embodiment 1 or embodiment 2, wherein the subject is at least 6 years of age and less than 10 years of age.

Embodiment 4. The method of any one of embodiments 1-3, wherein the subject is administered between about 5 mg and about 30 mg DSP.

Embodiment 5. The method of any one of embodiments 1-3, wherein the subject is administered between about 5 mg and about 22 mg DSP.

Embodiment 6. The method of any one of embodiments 1-5, wherein the subject is administered about 8 mg or about 17 mg DSP.

Embodiment 7. The method of any one of embodiments 1-5, wherein the subject is administered about 8 mg or about 17 mg DSP.

Embodiment 8. The method of any one of embodiments 1-7, wherein the subject is administered the DSP monthly, every 5 weeks, every 6 weeks, every 7 weeks or every two months.

Embodiment 9. The method of any one of claims 1-8, wherein the DSP is administered by infusion.

Embodiment 10. The method of any one of embodiments 1-9, wherein the treatment period is 3 months, 6 months, 9 months, or 12 months.

Embodiment 11. The method of any one of embodiments 1-10, wherein the erythrocytes are autologous to the subject.

Embodiment 10. The method of any one of embodiments 1-11, wherein the erythrocytes are loaded with DSP by:
(i) swelling erythrocytes, optionally isolated from whole blood, using a first hypotonic solution;
(ii) further swelling the erythrocytes in step (i) with a second hypotonic solution that is more hypotonic that the first hypotonic solution, wherein the further swelling is done without reaching lysis;
(iii) concentrating the erythrocytes obtained in step (ii);
(iv) placing the concentrated erythrocytes in contact with a lysing solution comprising, or alternatively consisting essentially of, or yet further consisting of DSP; and
(v) adding a sealing solution and thereby obtaining a population of erythrocytes loaded with DSP.

Embodiment 13. The method of any one of embodiments 1-11, wherein the erythrocytes are loaded with DSP by:
(a) removing whole blood from the subject;
(b) using the whole blood, preparing a population of erythrocytes by
   (i) isolating erythrocytes from the whole blood;
   (ii) swelling the erythrocytes using a first hypotonic solution;
   (iii) further swelling the erythrocytes in step (ii) with a second hypotonic solution that is more hypotonic that the first hypotonic solution, wherein the further swelling is done without reaching lysis;
   (iv) concentrating the erythrocytes obtained in step (iii);
   (v) placing the concentrated erythrocytes in contact with a lysing solution comprising DSP; and
   (vi) adding a sealing solution for the purpose of obtaining a population of erythrocytes loaded with said one or more pharmaceutical products.

Embodiment 14. The method of any one of embodiments 1-11, wherein the erythrocytes are loaded with DSP by:
(a) separating erythrocytes from the whole blood from 20-100 mL of whole blood;
(b) swelling the erythrocytes using a first hypotonic saline solution having an osmolality between 230 and 150 mOsm/kg;
(c) further swelling the erythrocytes using a second hypotonic solution having a lower osmolality than the first hypotonic saline solution, wherein the further swelling is done without reaching lysis, thereby providing swollen erythrocytes;
(d) contacting the swollen erythrocytes with a solution comprising between 2-10 mL of a solution comprising DSP at a concentration of 25 mg/mL; and
   incubating the swollen erythrocytes with a hypertonic sealing solution comprising phosphate-inosine-glucose-pyruvate-adenine (PIGPA).

Embodiment 15. The method of any one of embodiments 1-11, wherein the erythrocytes are loaded with DSP by:
(a) obtaining between 20-100 mL of whole blood of the subject;
(b) separating erythrocytes from the whole blood;
(c) swelling the erythrocytes using a first hypotonic saline solution having an osmolality between 230 and 150 mOsm/kg;
(d) further swelling the erythrocytes using a second hypotonic solution having a lower osmolality than the first hypotonic saline solution, wherein the further swelling is done without reaching lysis, thereby providing swollen erythrocytes;
(e) contacting the swollen erythrocytes with a solution comprising between 2-10 mL of a solution comprising DSP at a concentration of 25 mg/ml; and
   incubating the swollen erythrocytes with a hypertonic sealing solution comprising phosphate-inosine-glucose-pyruvate-adenine (PIGPA).

Embodiment 16. The method of embodiment 14 or embodiment 15, wherein the second hypotonic solution brings the intact erythrocytes to an osmolality between 200 and 170 mOsm/Kg.

Embodiment 17. The method of any one of embodiments 14 to 16, wherein about 50 mL of the whole blood is obtained from the subject.

Embodiment 18. The method of any one of embodiments 14 to 16, wherein about 30 mL of the whole blood is obtained from the subject.

Embodiment 19. The method of embodiment 18, wherein the subject is between 9 and 15 kilograms in weight.

### Equivalents

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

What is claimed is:

1. A method at treating Ataxia Telangiectasia in a subject less than about 10 years of age, comprising administering to the subject at least 5 mg of dexamethasone sodium phosphate (DSP) that has been loaded into erythrocytes.

2. A method at treating Ataxia Telangiectasia in a subject less than about 10 years of age, comprising administering to the subject at least 5 mg of dexamethasone sodium phosphate (DSP) that has been loaded into erythrocytes.

3. The method of claim 1, wherein the subject is at least 6 years of age and less than 10 years of age.

4. The method of claim 1, wherein the subject is administered between about 5 mg and about 30 mg DSP.

5. The method of claim 1, wherein the subject is administered between about 5 mg and about 22 mg DSP.

6. The method of claim 1, wherein the subject is administered about 8 mg or about 22 mg DSP.

7. The method of claim 1, wherein the subject is administered about 8 mg or about 17 mg DSP.

8. The method of claim 1, wherein the subject is administered the DSP monthly, every 5 weeks, every 6 weeks, every 7 weeks or every two months.

9. The method of claim 1, wherein the DSP is administered by infusion.

10. The method of claim 1, wherein the treatment period is 3 months, 6 months, 9 months, or 12 months.

11. The method of claim 1, wherein the erythrocytes are autologous to the subject.

12. The method of claim 1, wherein the erythrocytes are loaded with DSP by:
(i) swelling erythrocytes, optionally isolated from whole blood, using a first hypotonic solution;
(ii) further swelling the erythrocytes in step (i) with a second hypotonic solution that is more hypotonic that the first hypotonic solution, wherein the further swelling is done without reaching lysis;
(iii) concentrating the erythrocytes obtained in step (ii);
(iv) placing the concentrated erythrocytes in contact with a lysing solution comprising, or alternatively consisting essentially of, or yet further consisting of DSP; and
(v) adding a sealing solution and thereby obtaining a population of erythrocytes loaded with DSP.

13. The method of claim 1, wherein the erythrocytes are loaded with DSP by:
(a) removing whole blood from the subject;
(b) using the whole blood, preparing a population of erythrocytes by
(i) isolating erythrocytes from the whole blood;
(ii) swelling the erythrocytes using a first hypotonic solution;
(iii) further swelling the erythrocytes in step (ii) with a second hypotonic solution that is more hypotonic that the first hypotonic solution, wherein the further swelling is done without reaching lysis;
(iv) concentrating the erythrocytes obtained in step (iii);
(v) placing the concentrated erythrocytes in contact with a lysing solution comprising DSP; and
(vi) adding a sealing solution for the purpose of obtaining a population of erythrocytes loaded with said one or more pharmaceutical products.

14. The method of claim 1, wherein the erythrocytes are loaded with DSP by:
(a) separating erythrocytes from the whole blood from 20-100 mL of whole blood;
(b) swelling the erythrocytes using a first hypotonic saline solution having an osmolality between 230 and 150 mOsm/kg;
(c) further swelling the erythrocytes using a second hypotonic solution having a lower osmolality than the first hypotonic saline solution, wherein the further swelling is done without reaching lysis, thereby providing swollen erythrocytes;
(d) contacting the swollen erythrocytes with a solution comprising between 2-10 mL of a solution comprising DSP at a concentration of 25 mg/mL; and
(e) incubating the swollen erythrocytes with a hypertonic sealing solution comprising phosphate-inosine-glucose-pyruvate-adenine (PIGPA).

15. The method of claim 1, wherein the erythrocytes are loaded with DSP by:
(a) obtaining between 20-100 mL of whole blood of the subject;
(b) separating erythrocytes from the whole blood;
(c) swelling the erythrocytes using a first hypotonic saline solution having an osmolality between 230 and 150 mOsm/kg;
(d) further swelling the erythrocytes using a second hypotonic solution having a lower osmolality than the first hypotonic saline solution, wherein the further swelling is done without reaching lysis, thereby providing swollen erythrocytes;
(e) contacting the swollen erythrocytes with a solution comprising between 2-10 mL of a solution comprising DSP at a concentration of 25 mg/mL; and
incubating the swollen erythrocytes with a hypertonic sealing solution comprising phosphate-inosine-glucose-pyruvate-adenine (PIGPA).

16. The method of claim 14, wherein the second hypotonic solution brings the intact erythrocytes to an osmolality between 200 and 170 mOsm/Kg.

17. The method of claim 15, wherein the second hypotonic solution brings the intact erythrocytes to an osmolality between 200 and 170 mOsm/Kg.

18. The method of claim 14, wherein about 50 mL of the whole blood is obtained from the subject.

19. The method of claim 14, wherein about 30 mL of the whole blood is obtained from the subject.

20. The method of claim 19, wherein the subject is between 9 and 15 kilograms in weight.
